(19) United States
(12) Patent Application Publication
(10) Pub. No.: US 2026/0240845 A1
(43) Pub. Date: Aug. 20, 2026

(54) METHODS FOR TREATING OR PREVENTING CORONAVIRUS INFECTION SKELETAL SEQUELAE

(71) Applicants: The Hong Kong Polytechnic University, Hong Kong (CN); University of Hong Kong Versitech Limited, Hong Kong (CN)

(72) Inventors: Chunyi WEN, Hong Kong (CN); Shuofeng YUAN, Hong Kong (CN); Man Ting AU, Hong Kong (CN); Junguo NI, Hong Kong (CN)

(21) Appl. No.: 19/486,434

(22) PCT Filed: Feb. 22, 2024
(86) PCT No.: PCT/CN2024/078113

§ 371 Date: Nov. 21, 2025

Related U.S. Application Data
(60) Provisional application No. 63/448,532, filed on Feb. 27, 2023.

(51) Int. Cl. A61K 31/506; A61P 31/14

(52) U.S. Cl. CPC A61K 31/506; A61P 31/14

(57) ABSTRACT

Methods for treating or preventing a skeletal sequelae of a coronavirus infection in a subject in need thereof, the method comprising administering a therapeutically effective amount of an endothelin receptor antagonist to the subject, with the proviso that the endothelin receptor antagonist is not co-administered with dapagliflozin or niclosamide. The skeletal sequelae can include one or more of arthralgia, viral arthritis, osteopenia, osteochondral damage, and osteoporosis.

### CROSS-REFERENCE TO RELATED APPLICATIONS

This application claims the benefit of priority of U.S. Provisional Patent Application No. 63/448,532 filed Feb. 27, 2023, the contents of it being hereby incorporated by reference in its entirety for all purposes.

### TECHNICAL FIELD

The present disclosure relates to methods and compounds useful for treating coronavirus infection skeletal sequalae.

### BACKGROUND

Musculoskeletal disorders are a major post-acute sequelae of COVID-19 (PASC)¹. Among them, arthralgia, i.e., joint pain, is one of the most refractory long-haul symptoms³⁻⁶. There were 10-23% of hospitalised COVID survivors suffering from arthralgia at 2~3 months post-SARS-COV-2 infection^{7,8}. Without timely intervention, around 10% of them eventually developed debilitating long COVID pain syndrome up to one-year post-infection^{1,4}.

Virus infection is a known cause of joint pain and arthritis⁹. Respiratory virus infection has been associated with an increased risk of inflammatory arthritis¹⁰. Mounting clinical cases reported viral arthralgia¹¹ and inflammatory arthritis¹²⁻¹⁵ after SARS-COV-2 infection. SARS-COV-2 infection might cause joint damage, possibly via triggering hyperinflammation¹⁶ or autoimmune response¹⁷. Yet the exact molecular mechanism remains unknown.

Sustained elevation of endothelial dysfunction biomarkers^{18, 19}, e.g. endothelin-1 (ET-1)—the most potent vasoconstrictor^{20, 21}, was detected in the plasma of hospitalised COVID patients. Elevated plasma ET-1 was associated with the severity of joint damage in both humans^{22, 23} and animals²⁴. Elevated ET-1 has been shown to induce joint pain^{25, 26} and cartilage degradation²⁷ via its two G protein-coupled receptors, i.e., endothelin type A and B receptors (ETAR and ETBR). Blockade of endothelin receptors exerts analgesic and chondroprotective effects in a variety of arthritis animal models^{24, 28-30}. All the evidence suggests a plausible role of endothelin system in SARS-CoV-2-induced joint pain and damage.

There is thus a need to develop improved methods for treating corona virus infection skeletal sequelae.

### SUMMARY

The pathological manifestations of acute SARS-COV-2 infection has been recapitulated in a golden Syrian hamster model³¹. Pathological bone loss was observed in this model². Yet damage caused by SARS-COV-2 infection to articular cartilage capping bone ends remains under investigation.

Herein, the temporal changes of joint pathology after SARS-COV-2 infection using this well-established hamster model³¹ are characterized. Osteochondral damage was observed in the acute phase of infection with activation of endothelin signalling. We further demonstrated timely intervention using an endothelin receptor blocker, such as macitentan, could alleviate joint damage caused by viral infection. Initiation of endothelin receptor blocker treatment even in the sub-acute phase of infection was still able to restore the structural integrity of subchondral bone.

In a first aspect, provided herein is a method of treating or preventing a skeletal sequelae of a coronavirus infection in a subject in need thereof, the method comprising administering a therapeutically effective amount of an endothelin receptor antagonist to the subject, with the proviso that the endothelin receptor antagonist is not co-administered with dapagliflozin or niclosamide.

In certain embodiments, the subject does not suffer from pulmonary hypertension.

In certain embodiments, the coronavirus infection is the result of a coronavirus selected from the group consisting of severe acute respiratory syndrome coronavirus (SARS-COV), severe acute respiratory syndrome coronavirus 2 (SARS-COV-2), Middle East respiratory syndrome-related coronavirus (MERS-COV), human coronavirus 229E (229E-CoV-2), human coronavirus NL63 (NL63-CoV), human coronavirus OC43 (OC43-CoV), human coronavirus HKU1 (HCoV-HKU1), and variants thereof.

In certain embodiments, the coronavirus infection is the result of a severe acute respiratory syndrome coronavirus 2 (SARS-COV-2) and variants thereof.

In certain embodiments, the endothelin receptor antagonist is a selective ETA receptor antagonist, a selective ETB receptor antagonist, or a dual ETA receptor and ETB receptor antagonist.

In certain embodiments, the endothelin receptor antagonist is a dual ETA receptor and ETB receptor antagonist.

In certain embodiments, the endothelin receptor antagonist is selected from the group consisting of sitaxsentan, ambrisentan, atrasentan, BQ-123, sparsentan, zibotentan, aprocitentan, edonentan, bosentan, macitentan, tezosentan, BQ-788, A192621, and mixtures thereof.

In certain embodiments, the endothelin receptor antagonist is bosentan, macitentan, ambrisentan, or a mixture thereof.

In certain embodiments, the endothelin receptor antagonist is macitentan.

In certain embodiments, the coronavirus infection is the result of a severe acute respiratory syndrome coronavirus 2 (SARS-COV-2) and the endothelin receptor antagonist is bosentan, macitentan, ambrisentan, or a mixture thereof.

In certain embodiments, the endothelin receptor antagonist is administered to the subject in at least one of the coronavirus infection acute phase or coronavirus infection sub-acute phase.

In certain embodiments, the skeletal sequelae is one or more of arthralgia, viral arthritis, osteopenia, osteochondral damage, and osteoporosis.

In certain embodiments, the endothelin receptor antagonist is administered to the subject intraarticular injection or intraosseous injection.

In certain embodiments, the coronavirus infection is the result of a severe acute respiratory syndrome coronavirus 2 (SARS-COV-2) and the endothelin receptor antagonist is macitentan.

In certain embodiments, macitentan is administered to the subject in at least one of SARS-COV-2 acute phase or coronavirus infection sub-acute phase.

In certain embodiments, the skeletal sequelae is one or more of arthralgia, viral arthritis, osteopenia, osteochondral damage, and osteoporosis.

### BRIEF DESCRIPTION OF THE DRAWINGS

The above and other objects and features of the present disclosure will become apparent from the following description of the disclosure, when taken in conjunction with the accompanying drawings.

FIG. 1 |SARS-COV-2 infection induces osteochondral damage in hamsters. a, Experimental design to study the time course changes of joints after SARS-COV-2infection using a golden Syrian hamster model. Fifteen hamsters were used for this aim. Ten were infected with SARS-COV-2 wildtype by intranasal administration.

Five underwent mock infection. Hind limbs were collected at designated time points. n=5 for each time point. b, Representative micro-CT images and corresponding measurements [bone mineral density (BMD) and trabecular bone volume fraction (BV/TV) ] in the proximal epiphysis of tibia in infected hamsters. n=5 per time point. c, Representative TRAP staining and its respective quantification (N. Oc/B. Pm) at 4 and 30 dpi. Scale bars, 50 μm. d, Representative SP7 staining and its respective quantification showing time-course changes of SP7⁺ osteoblasts per bone perimeter (N. Ob/B. Pm). Scale bars, 25 μm. e, f, Representative (e) H&E and (f) Masson's Trichrome (MT) images of articular cartilage to show cysts formation (arrowhead), fibrin exudate (arrow) and collagen deposition after SARS-COV-2 infection. Scale bars, 100 μm (H&E); 50 μm (MT). g, h, Quantitative evaluation of (g) cyst area and (h) number of chondrocytes in articular cartilage. i, Representative safranin O/fast green staining and its respective intensity quantification showing loss of proteoglycan in cartilage after virus infection. Scale bars, 100 μm. j-l Representative immunostaining images and quantification for (j) p16^{INK4a}, (k) HMGB1 and (l) caspase 3 in mock-infected group and different time points after infection. Scale bars, 50 μm (j, k) ; 100 and 20 μm (l). Data are mean ±S. E. M. Data were excluded when they fall out of 1.5 IQR. One-way ANOVA with Tukey's post hoc test.

FIG. 2 |SARS-COV-2 infection triggers endothelial dysfunction with systemic activation of endothelin signalling. a, b, Representative images of (a) CellROX and (b) MitoTracker staining showing accumulation of oxidative stress and changes in mitochondrial dynamics in HUVEC after pV D614G (MOI of 10) and SP RBD (10 μg/ml) treatment for 1 hour. Hydrogen peroxide at 100 μM was used as positive control. Blob form structures were indicated by arrows. Scale bars, (a) 50 μm; (b) 10 μm. c, d, Relative mRNA expression of (c) EDN 1, (d) endothelin receptors and RANKL after pV or SP RBD stimulation for 24 h in HUVEC. Two-tailed Student's t-test e, Serum ET-1 level of hamsters underwent mock infection (n=4), 4 dpi (n=5) or 30 dpi (n=4) after SARS-COV-2 infection measured by ELISA. One-way ANOVA followed by Tukey's post hoc test. Data were excluded when they fall out of 1.5 IQR. f, Representative images of multichannel fluorescent staining showing the temporal expression pattern ETAR and ETBR knee articular cartilage of hamsters underwent mock or SARS-COV-2 infection. Nuclei were counterstained using SN520. Scale bars, 50 μm. Quantification was done by DAB staining. Mock (n=5); 4 dpi (n=4); 30 dpi (n=5). g, Representative immunofluorescent staining of vWF and 4HNE in subchondral bone marrow cavity of proximal tibia in hamsters. Co-localisation of vWF and 4HNE was indicated by an arrow. Nuclei were counterstained with DAPI. Scale bars, 10 μm.

FIG. 3 |Viral spike causes chondrocyte oxidative stress and senescence with activation of endothelin signalling. a, b, Representative images of (a) SARS-COV-2 nucleocapsid protein (NP) and (b) SARS-COV-2 spike protein (SP) in subchondral bone at different time points after infection. n=5 per time point. Scale bars, 25 μm. Semi-quantification of NP and SP was performed based on integrated optical density (IOD). c, d, Relative mRNA expression of (c) EDN1 and its receptors and (d) matrix metalloproteinases and p16^{INK4a} in chondrocyte cell line C28/12 24 h upon 10 μg/ml SARS-COV-2 spike protein RBD (SP RBD) or ET-1 (100 nM) stimulation. (c) Two-tailed Student's t-test; (d) One-way ANOVA with Tukey's post hoc test. Data are mean±S. E. M. e, Representative immunofluorescence staining for p16^{INK4a} of chondrocyte cell line ATDC 5 with and without SP RBD challenge for 24 h. n=3. Scale bar, 50 ml. f, Representative images of SA-β-gal staining on ATDC5 cells with and without SP RBD stimulation. n=3. g, h, Representative images of (g) CellROX and (h) MitoTracker staining showing accumulation of oxidative stress and changes in mitochondrial dynamics in C 28/12 after SP RBD (10 μg/ml) treatment for 1 hour. C28/12 cells were pre-treated with macitentan for 30 minutes before stimulation by SP RBD for 1 h. Scale bars, (g) 50 ml; (h) 25 ml.

FIG. 4 |Viral spike contributes to joint pain and chondrocyte senescence. a, Experimental design showing mouse model intravenously injected with SARS-COV-2 spike protein RBD (10 μg in 150 μl saline) and the treatment efficacy of Macitentan. Macitentan treatment at 2 doses was started 2 days after RBD injection and lasted for 12 days. The animals were sacrificed 14 days after injection of RBD. Vehicle (n=3) ; SP RBD (n=4), Low Mac (n=3) ; High Mac (n=4). b, Time-course changes of paw withdrawal threshold evaluated by von Frey filament test in response to mechanical stimuli in mice with or without SP RBD injection. Two-tailed Student's t-test. c, Effect of macitentan treatment at two different dosages (0.3 mg/kg and 3 mg/kg) on paw withdrawal threshold in mice injected with SP RBD. Two-way ANOVA with Tukey post hoc correction for multiple testing. d, Representative micro-CT images of the proximal epiphysis of tibias showing the rescue effect of macitentan on SP RBD-induced bone loss. e, Representative Safranin O/fast green staining showing loss of proteoglycan in joint articular cartilage in mice with SP RBD. Scale bars, 200 μm. f, Representative immunostaining for ET-1 in articular cartilage in mice with and without macitentan treatment. Scale bars, 50 μm. g, h, Representative immunostaining for (g) p16^{INK4a} and (h) HMGB1 in articular cartilage showing accumulation of senescent chondrocytes with and without macitentan treatment in SP RBD-injected mice. Haematoxylin and DAPI were used as counterstains in (g) and (h) respectively.

FIG. 5 |Early macitentan treatment partially rescues SARS-COV-2-associated osteochondral damage. a, Experimental design showing the treatment regimen using Macitentan at 0.3 mg/kg in SARS-COV-2-infected hamsters for 4 days from 1 dpi. PBS was used as vehicle control. Animals were sacrificed at 4 dpi, hind limbs were harvested and analysed. 4 dpi PBS (n=5); 4 dpi Mac (n=4) b, Representative immunostaining for SARS-CoV-2 spike protein (SP) in subchondral bone marrow cavity with and without macitentan treatment at acute phase of infection. Scale bars, 25 μm. c, Representative multi-channel immunofluorescent staining for vWF and 4HNE in subchondral bone marrow cavity of proximal tibia of hamsters to show improvement of vascular function with early macitentan treatment. Co-localisation of vWF and 4HNE was indicated by an arrow. DAPI was used as counterstain. Scale bars, 25 μm; 10 μm (magnified). d, e, Representative TRAP and SP7 staining and the respective quantifications. Scale bars, 50 μm. f, Micro-CT images of proximal epiphysis of tibias of hamsters showing changes in BMD and BV/TV with early macitentan treatment. g, h, Histopathological analysis of knee articular cartilage of infected hamsters using (g) H&E and (h) Masson's trichrome staining. Scale bars, (g) 100 μm; (h) 50 μm. i, j, Quantitative evaluation of (i) cyst area and (j) number of chondrocytes in articular cartilage with or without macitentan treatment at acute phase of infection. k, l, Representative immunofluorescent staining and quantification for (k) p16^{INK4a} and (l) HMGB1 in articular cartilage. Nuclei were stained using DAPI. Scale bars, 50 μm. Two-tailed Student's t-test. Data were excluded when they fall out of 1.5 IQR.

FIG. 6 |Delayed macitentan treatment alleviates subchondral bone loss at sub-acute phase of SARS-COV-2 infection. a, Schematic diagram showing the experimental plan to study the effect of delayed macitentan treatment on SARS-COV-2-infected hamsters. Macitentan at 0.3 mg/kg was administered intraperitoneally from 16 dpi to 30 dpi daily. PBS was used as vehicle control. Animals were euthanised at 30 dpi, hind limbs were collected and analysed. n=5 for each group. b, Representative immunostaining for SARS-COV-2 spike protein (SP) in subchondral bone marrow cavity with and without macitentan treatment at sub-acute phase of infection. Scale bars, 25 μm. c, Representative multi-channel immunofluorescent staining for vWF and 4HNE in subchondral bone marrow cavity of proximal tibia of hamsters to show reduction of oxidative stress near vessels with delayed macitentan treatment. DAPI was used as a counterstain. Scale bars, 25 μm; 10 μm (magnified). d, Micro-CT images of proximal epiphysis of tibias of hamsters showing significant increase in BMD and BV/TV with delayed macitentan treatment. e, f, Representative SP7 and TRAP staining and the respective quantifications. Scale bars, 50 μm. g, h, Representative images of knee articular cartilage of infected hamsters using (g) H&E and (h) Masson's trichrome staining. Scale bars, (g) 100 μm; (h) 50 μm. i, j, Quantitative evaluation of (i) cyst area and (j) number of chondrocytes in articular cartilage with or without macitentan treatment at sub-acute phase of infection. n=5. k, l, Representative immunofluorescent staining and quantification for (k) p16^{INK4a} and (l) HMGB1 in articular cartilage. DAPI was used as counterstain. Scale bars, 50 μm. n=5. Unpaired two-tailed Student's t-test. Data were excluded when they fall out of 1.5 IQR.

FIG. 7 |Subchondral bone microstructural deterioration in proximal epiphysis of tibia in hamsters infected by SARS-COV-2. Measurement of trabecular thickness (Tb. Th), number (Tb. N.) and separation (Tb. S) of proximal epiphysis of tibia in golden Syrian hamster underwent SARS-COV-2 infection 4-and 30-day post-infection in comparison to mock infection group. n=5 for each group. Data was presented as mean ±S. E. M. One-way ANOVA with Tukey's multiple-comparison.

FIG. 8 |No obvious morphological changes in meniscus and synovium before and after SARS-COV-2 infection. Representative H&E staining showed no significant abnormal pathological changes in meniscus and synovium (meniscal extrusion and tear, infiltration of mononuclear cells, thickening of synovial linings) in hamsters infected by SARS-COV-2 when compared to mock infection group.

FIG. 9 |No obvious changes in articular cartilage surface roughness. a, The H&E images of articular cartilage were cropped from original images for analysis. b, The images were adjusted and binarized for analysis. The contour of articular surface was extracted using the findContours function in OpenCV. Then the roughness of articular surface was calculated using the length of articular surface divided by its width based on previously published protocol. c, No statistical significance in terms of articular surface roughness after SARS-COV-2 infection and macitentan treatment.

FIG. 10 |Semi-quantification of SARS-COV-2 viral components. a, Grading of the presence of SARS-COV-2 nucleocapsid protein (NP) in bone marrow cavity in subchondral bone region in tibias of hamsters based on integrated optical density (IOD) in per-mille (‰). IODs of all sections were divided into 3 categories from “−”, indicating the lowest abundance, to “++”, indicating the highest abundance. b, Table showing the number of samples in each grade based on IOD. Chi-square test. c, Grading of the presence of SARS-COV-2 spike protein (SP) in bone marrow cavity in subchondral bone region in tibias of hamsters based on integrated optical density (IOD) in percent (%). IODs of all sections were divided into 4 categories from “−”, indicating the lowest abundance, to “+++”, indicating the highest abundance. b, Table showing the number of samples in each grade based on IOD. Mock (n=5); 4 dpi PBS (n=5); 4 dpi Mac (n=4); 30 dpi PBS (n=5) and 30 dpi Mac (n=5). Chi-square test.

FIG. 11 |SARS-COV-2 spike protein RBD causes deterioration of subchondral bone structural integrity in mice. Measurement of bone volume fraction (BV/TV), trabecular thickness (Tb. Th), number (Tb. N.) , separation (Tb. S) and structure model index (SMI) of the tibial epiphysis in mice underwent different treatment. Vehicle control (n=4); SP RBD (n=4); Low dose Mac (0.3 mg/kg) (n=4) and High dose Mac (3 mg/kg) (n=5). Data was presented as mean±S.E.M. Mann-Whitney test was used in comparison between vehicle control and SP RBD.

FIG. 12 |Effect of early macitentan treatment on subchondral bone microstructure. Measurement of trabecular thickness (Tb. Th), number (Tb. N.) and separation (Tb. S) of proximal epiphysis of tibia in golden Syrian hamster with or without macitentan treatment 4 dpi after infection. Data was presented as mean±S.E.M. 4 dpi PBS (n=5) ; 4 dpi Mac (n=4). Unpaired two-tailed Student's t-test.

FIG. 13 |Effect of delayed macitentan treatment on subchondral bone microstructure. Measurement of trabecular thickness (Tb. Th), number (Tb. N.) and separation (Tb. S) of proximal epiphysis of tibia in golden Syrian hamster with or without macitentan treatment 30 dpi after infection. Data was presented as mean±S.E.M. 30 dpi PBS (n=5) and 30 dpi Mac (n=5). Unpaired two-tailed Student's t-test.

### DETAILED DESCRIPTION

### Definitions

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0%variation from the nominal value unless otherwise indicated or inferred.

The term “therapeutically effective amount” as used herein, means that amount of the compound or therapeutic agent that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

As used herein, the term “sequelae” refers to one or more pathological conditions resulting from a coronavirus infection in a subject.

As used herein, the term “subject” refers to any animal (e.g., a mammal), including, but not limited to, humans, non-human primates, canines, felines, and rodents.

As used herein, the term “variant” when used in connection with a virus (such as, SARS-COV-2) means a virus that is a progeny of a reference (or “parent”) virus that possesses one or more changes in its genome (e.g., a RNA genome), or a virus that is genetically engineered to have one or more changes in its genome, relative to a reference (or “parent”) virus, which may or may not result in changes to the proteins encoded by the RNA sequence (e.g., one or more proteins of a variant virus may include substitutions, deletions, or insertions compared to a parent strain). For example, known variants of SARS-COV-2 include, but are not limited to, B. 1.1.7 (alpha variant), B. 1.351 (beta variant), P. 1 (gamma variant), B. 1.617.2 (delta variant), and B. 1.1.529 (omicron variant). A variant of a virus may comprise a genome sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to about 100% sequence identity or homology with the reference (or “parent”) genome sequence.

As used herein the term “substantially silences EDNRB and/or EDNRA” means that expression or protein activity or level of the target gene or protein encoded by the target gene in the presence of the introduced inhibitory nucleic acid molecule targeting a EDNRB and/or EDNRA nucleic acid sequence is reduced by about 10% to 100%, 10% to 90%, 20%to 90%, 30% to 90%, 40% to 90%, 50% to 90%, 50% to 80%, 50% to 70%, 55% to 70%, 55% to 69.2%, 70% to 90%, or 80% to 90% as compared to the expression or protein activity or level of the target gene or protein encoded by the target gene seen when the inhibitory nucleic acid molecule targeting a endothelin-1 nucleic acid sequence is not present. Generally, when a gene is substantially silenced, it will have at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or even 100% reduction in expression or protein activity or level of the target gene or protein encoded by the target gene as compared to when the inhibitory nucleic acid molecule is not present.

method of treating or preventing a skeletal sequelae of a coronavirus infection in a subject in need thereof, the method comprising administering a therapeutically effective amount of an endothelin receptor antagonist to the subject.

In certain embodiments, the endothelin receptor antagonist is not co-administered with a human renal sodium-dependent glucose transporter 2 (SGLT2) inhibitor, such as dapagliflozin. In certain embodiments, the endothelin receptor antagonist is not co-administered with an anthelmintic, such as niclosamide.

In certain embodiments, the subject does not suffer from pulmonary arterial hypertension (PAH), cancer, cardiovascular disease, hypertension, pulmonary hypertension, diabetic nephropathy, renal disease, occlusive vascular disease, congestive heart failure, a brain haemorrhage, cerebral aneurysm, a glomerular disorder, nephropathy, congestive heart failure, or a connective tissue disorder.

The endothelin receptor antagonist can be an antibody, an antibody fragment (such as Fab, Fab′, F (ab′) 2, Fv, or single chain (ScFv)), a peptide, an inhibitory nucleic acid molecule, or a small molecule (synthetic or naturally isolated or derived), wherein the inhibitory nucleic acid molecule substantially silences EDNRB and/or EDNRA.

In certain embodiments, the endothelin receptor antagonist is a selective ET_{A} receptor antagonist, such as sitaxsentan, ambrisentan, atrasentan, zibotentan, darusentan, avosentan, and clazosentan, a selective ETB receptor antagonist, or a dual ET_{A} receptor and ET_{B} receptor antagonist, such as bosentan, tezosentan, and macitentan.

Exemplary the endothelin receptor antagonist include, but are not limited to, sitaxsentan, ambrisentan, atrasentan, BQ-123 (cyclo (-D-Trp-D-Asp-Pro-D-Val -Leu-)), sparsentan, zibotentan, aprocitentan, edonentan, bosentan, macitentan, tezosentan, BQ-788 (sodium N-{[(2R, 6S)-2, 6-dimethyl-1-piperidinyl] carbonyl}-4-methyl-L-leucyl-N-[(1R)-1-carboxylatopentyl]-1-(methoxycarbonyl)-D-tryptophanamide), A192621 ((2R, 3R, 4S)-4-(1, 3-benzodioxol-5-yl)-1-[2-[(2, 6-diethylphenyl) amino]-2-oxoethyl]-2-4-propoxyphenyl) pyrrolidine-3-carboxylic acid), and mixtures thereof.

In certain embodiments, the endothelin receptor antagonist is macitentan.

The endothelin receptor antagonist can be administered after possible exposure of a subject to a coronavirus, after development of one or more coronavirus infection symptoms, after a positive diagnosis of a coronavirus infection, during the coronavirus infection acute phase, during a coronavirus infection sub-acute phase, during a coronavirus infection recovery phase, during a coronavirus infection chronic phase, or a combination thereof.

The endothelin receptor antagonist can be administered parenterally or non-parentally. Parenteral administration includes intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraarticular, intraosseous, intraspinal and intrasternal injection and infusion. Non-parenteral administration includes oral, sublingual, topical, transdermal, ophthalmic, otic, nasal, vaginal, rectal, mucosal, or percutaneous. In certain embodiments, the endothelin receptor antagonist is administered by intravenous, intraarticular, or intraosseous injection.

In certain embodiments, the method further comprises co-administering a second therapeutic agent, such as an anti-viral agent, an anti-bacterial agent, an anti-parasitic agent, an analgesic, an anti-inflammatory, or a combination thereof. In certain embodiments, the antiviral agent is Paxlovid, Molnupiravir, sotrovimab, remdesivir, lopinavir, ritonavir, or a combination thereof. The anti-bacterial agent can be azithoromycin or ciproflaxin. The anti-inflammatory agent can be dexamethasone.

The coronavirus infection can be caused by an existing coronavirus, such as severe acute respiratory syndrome coronavirus (SARS-COV), severe acute respiratory syndrome coronavirus 2 (SARS-COV-2), Middle East respiratory syndrome-related coronavirus (MERS-COV), human coronavirus 229E (229E-CoV-2), human coronavirus NL63 (NL63-CoV), human coronavirus OC43 (OC43-CoV), and human coronavirus HKU1 (HCoV-HKU1) or novel variants thereof.

In certain embodiments, SARS-COV is wild type SARS-COV, B. 1.1.7 (alpha variant), B. 1.351 (beta variant), P. 1 (gamma variant), B. 1.617.2 (delta variant), B. 1.1.529 (omicron variant), or a novel variant thereof.

The subject can be asymptomatic or symptomatic. In instances in which the subject tis symptomatic, the subject can exhibit one or more symptoms selected from the group consisting of fever, cough, fatigue, shortness of breath, muscle pain, joint pain, sore throat, headache, and chills (e.g., fever, cough, and shortness of breath). In certain embodiments, the one or more symptoms can appear from 2-14 days after the subject's exposure to coronavirus.

The subject can optionally be diagnosed with the coronavirus infection prior to the step of administering the therapeutically effective amount of the endothelin-1 antagonist. The coronavirus can be diagnosed using any method known in the art including, but not limited to, polymerase chain reaction (PCR), such as reverse transcription PCR (RT-PCR), real-time PCR (e.g., quantitative PCR (qPCR) ), and real-time RT-PCR (rRT-PCR), or an antigen test, such as a lateral flow test.

The skeletal sequelae can comprise one or more of arthralgia, viral arthritis, osteopenia, osteochondral damage, and osteoporosis.

The arthralgia and/or the osteochondral damage can involve one or more joints selected from the group consisting of a sternoclavicular, a shoulder, an elbow, a metacarpophalangeal, a sacroiliac, a knee, a transverse tarsal, an acromioclavicular, a radiocarpal, a carpometacarpal, a hip, an ankle, an interphalangeal, a temporo-mandibular, a shoulder, a zygapophyseal, and a subtalar joint. In certain embodiments, the arthralgia involves a joint selected from the group consisting of an ankle joint, a knee joint, a sacroiliac joint, and a combination thereof.

The viral arthritis, the osteopenia, and/or the osteoporosis can involve one or more bones selected from the group consisting of a cervical vertebrae, a thoracic vertebrae, a lumbar vertebrae, sacrum, a coccyx, a sternum, a rib, an occipital bone, a parietal bone, a frontal bone, a temporal bone, an ethmoid bone, a sphenoid bone, a lacrimal bone, a maxillae bone, a nasal bone, a palatine bone, a zygomatic bone, an inferior nasal concha, a mandible, a hyoid bone, a vomer bone, a humerus bone, a radius bone, an ulna bone, a carpal bone, a phalange bone, a metacarpal bone, a femur bone, a tibia bone, a fibula bone, a patella bone, a tarsal bone, a phalange bone, a metatarsal bone, a clavicle bone, a scapula bone, or a hip bone. In certain embodiments, the viral arthritis involves one or more bones selected from the group consisting of a patella, fibula, a tibia, a femur, a vertebrae, and a combination thereof.

In certain embodiments, the subject is at least 40 years old, at least 50 years old, at least 60 years old, at least 70 years old, or at least 80 years old.

Discussion Here we reported the phenotype of SARS-COV-2-induced osteochondral damage in a hamster model although joint pain has already been recognised as a prominent post-acute COVID symptom in human. Currently, clinical diagnosis of viral or reactive arthritis post-SARS-COV-2 infection relies on the symptom, i.e., joint pain, and laboratory tests in case series^{13-15, 35, 43}, lacking concrete imaging evidence showing structural damage to the joint after viral infection. Unlike the typical features of arthritis, we only observed osteochondral damage in the infected hamsters without any obvious change in other joint tissue such as synovium and meniscus. Our finding prompts an urgent need to screen for structural damage in COVID survivors with sustained joint pain. Moreover, we shall raise public awareness of this debilitating long-haul post-COVID skeletal complication, which requires prompt attendance and timely intervention.

The pathomechanism of virus-induced osteochondral damage or arthritis remains controversial. It could be a result of direct viral insult³⁵, hyperinflammation¹⁶ or autoimmune response¹⁷. The SARS-COV-2 viral mRNA was once detected in the synovial fluid of COVID patients³⁵. We also found the presence of viral spike and nucleocapsid proteins in subchondral bone marrow cavity in infected hamsters. This line of evidence points to the direction of direct viral insult. In the meantime, we showed elevated plasma and endothelial ET-1 in response to SARS-COV-2 exposure. Our data suggest the synergistic effect of direct viral insult and hyperinflammation ET-1 to drive osteochondral damage. Our work further substantiates this notion.

Significant bone loss in long bones and lumbar vertebrae was observed in the golden Syrian hamster model after SARS-COV-2 infection². It was speculated that SARS-COV-2 triggers cytokine storm and the increased proinflammatory cytokines such as tumour necrosis factor-alpha (TNF-alpha) and interleukin-1 beta (IL-1beta) further induce osteoclastogenesis via upregulation of receptor activator of nuclear factor kappa-B ligand (RANKL) in lung epithelial cells. Here, our study suggested another proinflammatory cytokine candidate, ET-1, which was reported to stimulate osteoclastic activity and bone resorption via its type A receptor⁴⁴⁻⁴⁶. It implies endothelin type A receptor is a plausible therapeutic target for SARS-COV-2-induced subchondral bone loss.

SARS-COV-2 can trigger senescence in a variety of cells and tissues^{47, 48}, driving organ damage⁴⁹. Elimination of senescent cells has emerged as a promising therapeutic strategy for COVID-19-induced organ dysfunction^{49, 50}. The exact molecular mechanism of SARS-COV-2-induced cellular senescence is not fully understood. Viral spike has been recently reported to induce endothelial senescence directly⁵¹. We also found viral spike RBD could trigger chondrocyte senescence in our study. Apart from viral spike protein, ET-1 can accelerate cellular senescence. Blockade of endothelin type B receptor effectively prevented the accumulation of senescent cells in various types of cells and animal models^{24, 52, 53}. Therefore, we repurposed macitentan, an FDA-approved anti-hypertensive drug with dual ETAR and ETBR blockade function, for treating virus-induced organ damage, e.g. osteochondral junction in knee joint.

We demonstrated that macitentan could effectively rescue osteochondral damage in a pre-clinical setting, either in acute phase or sub-acute phase of SARS-COV-2 infection. In addition to the anti-inflammatory effect via endothelin receptor blockade⁵⁴, the potential antiviral effect of endothelin receptor antagonists has also been documented to lower influenza and cytomegalovirus viral RNA counts in vitro^{55, 56}. We observed macitentan could decrease viral spike protein in local joint tissue in infected hamsters in vivo. In line with these experimental results, a clinical case series study reported unexpected recovery of SARS-COV-2 pneumonia in the pulmonary hypertensive patients treated by endothelin receptor antagonists including macitentan37. A phase 2/3 clinical trial is ongoing to apply ambrisentan, an endothelin receptor antagonist, for hospitalised patients with COVID-19 to reduce multi-organ damage by improving vascular function (NCT04393246)⁵⁸. It warrants further investigation into the anti-viral effect of endothelin receptor antagonists in the context of COVID-19.

There are two major limitations in the present study that should be taken into careful consideration in data interpretation. First, the age of animals we used in this study was 10 to 12 weeks old which corresponds to the puberty period in female golden Syrian hamsters³⁹. It is equivalent to the adolescence stage in human when skeletal development is ongoing. However, due to limited healing capacity of articular cartilage⁴⁰, the number of chondrocytes cannot be replenished after viral infection. As known, senescent cells are more susceptible to virus infection⁵¹. It warrants further study into osteochondral damage in aged hamsters rather than young ones. Second, we investigated the wild type of SARS-COV-2 i.e., HKU-001a. The severity of osteochondral damage is estimated to vary greatly among different strains of SARS-CoV-2 variants. It prompts further comparative study in this direction.

Collectively, we delineated the time-course changes of bone-cartilage functional unit in response to SARS-COV-2 infection in hamsters. SARS-COV-2 causes cellular oxidative stress and endothelial dysfunction with systemic and local activation of endothelin signalling. ET-1 can enhance vascular permeability⁶⁰ and may allow viral proteins to leak from systemic bloodstream to local joint tissue. ET-1 can also trigger osteoclastogenesis to induce subchondral bone loss and cystic formation at osteochondral junction possibly via its type A receptor. It may promote chondrocyte senescence and dropout, and ultimately cartilage damage via type B receptor.

Dual blockade of both endothelin type A and B receptors e.g. using macitentan, can effectively alleviate subchondral bone loss and lower virus-induced articular chondrocyte damage. The findings generated from this study lay a solid scientific foundation to reposition endothelin receptor antagonists for management of long COVID joint damage and arthralgia.

### RESULTS

### SARS-COV-2 Infection Induces Osteochondral Damage in Hamsters

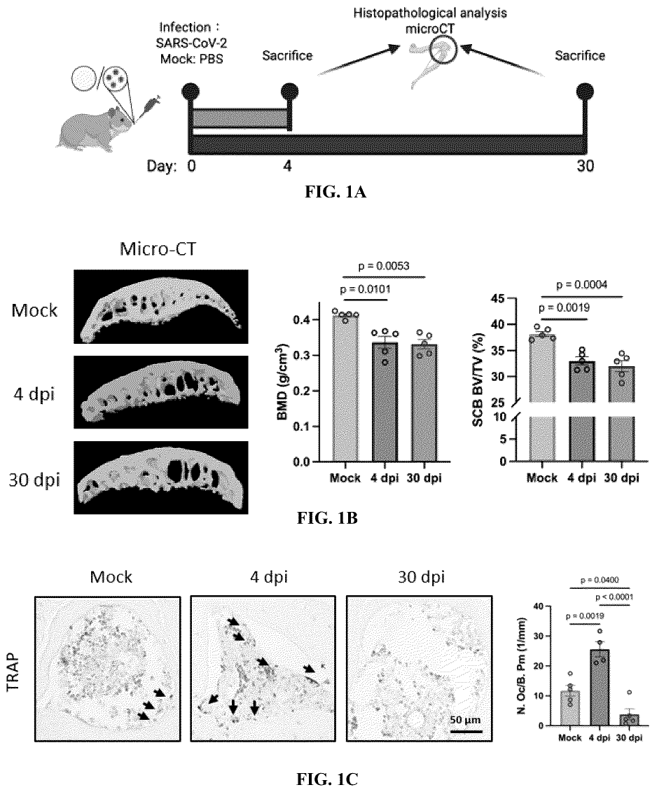

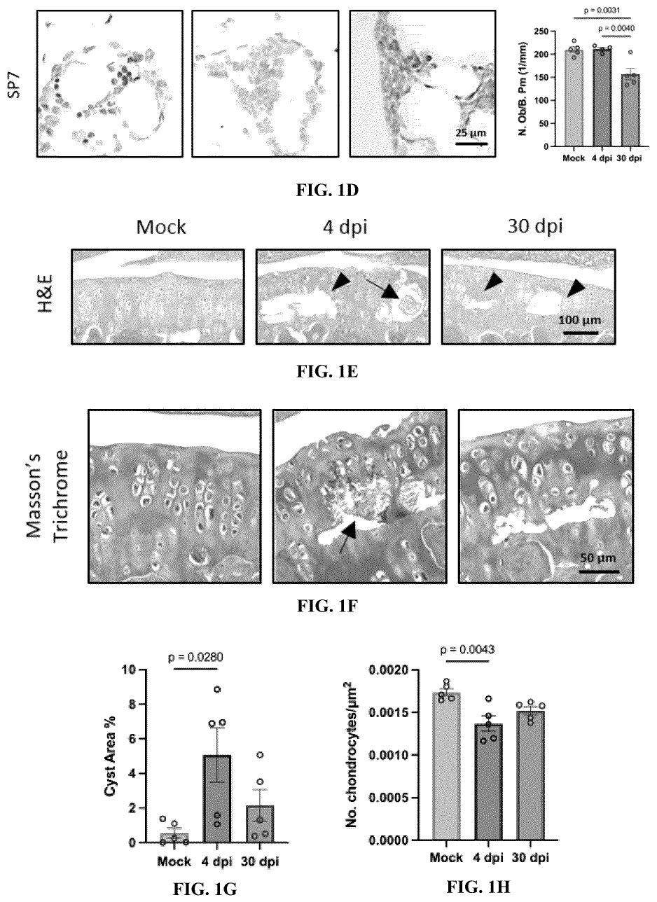

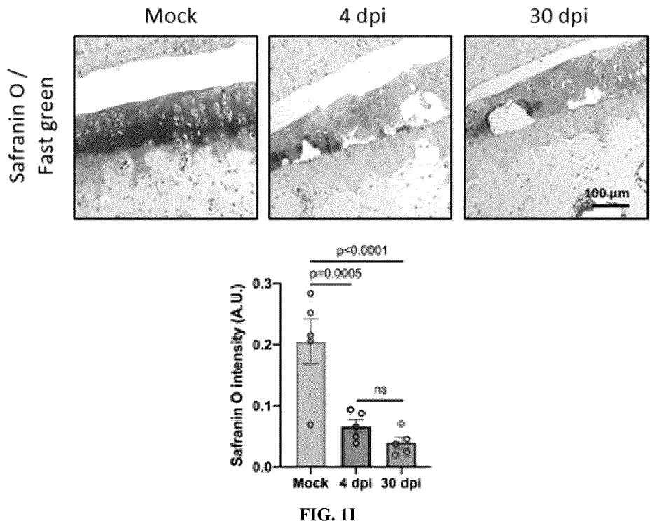

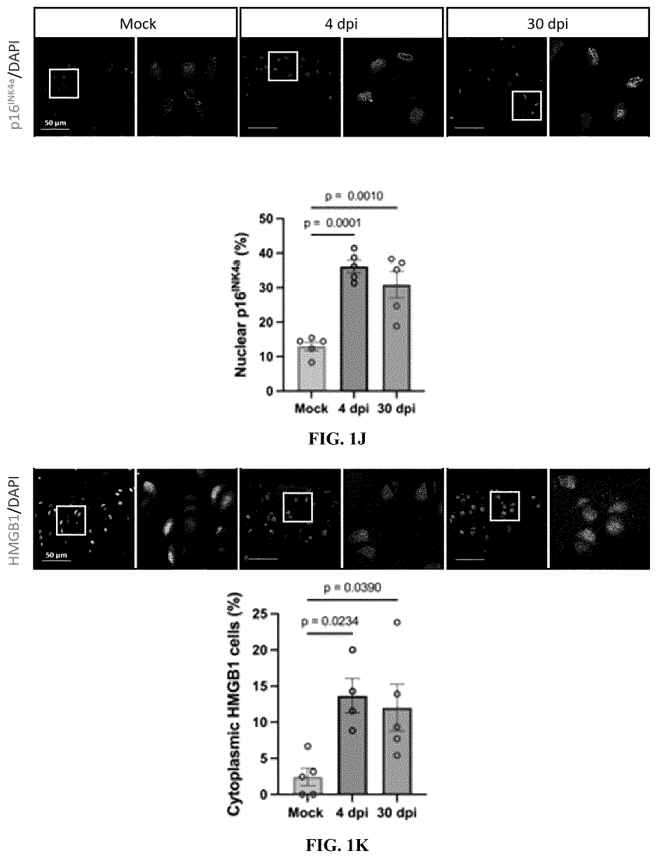

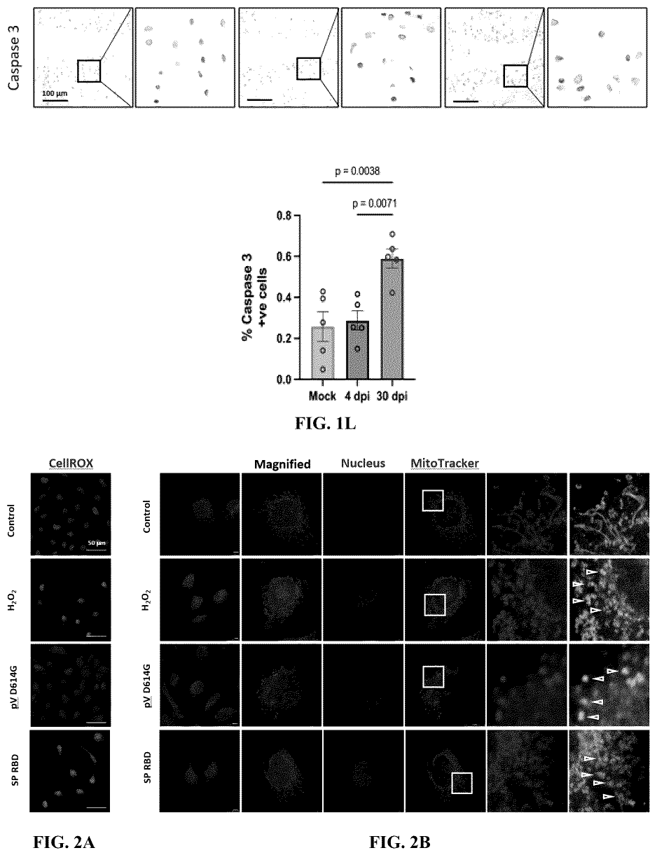

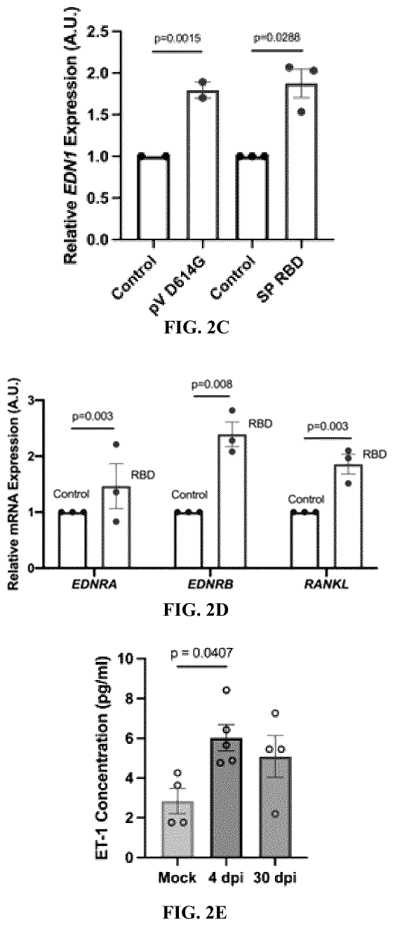

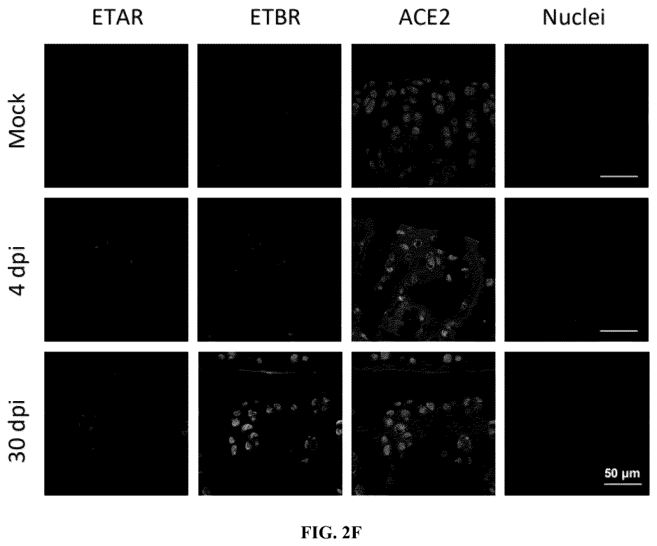

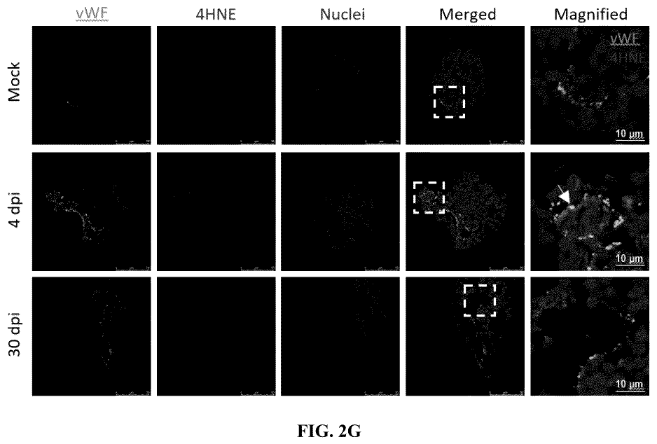

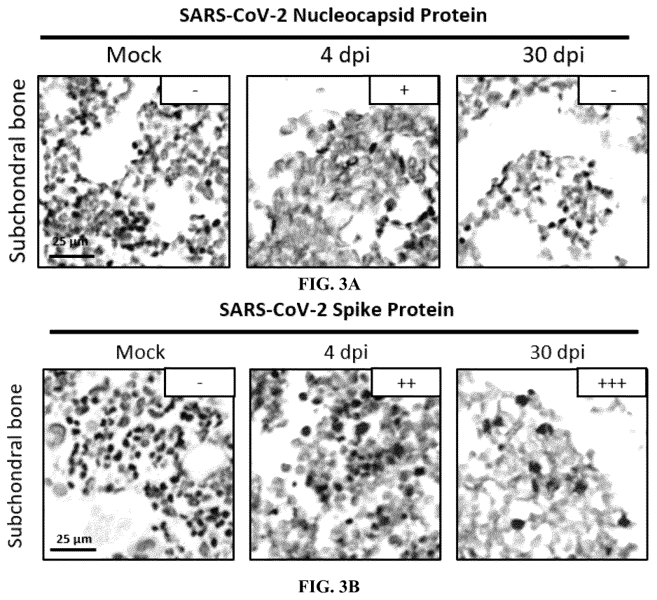

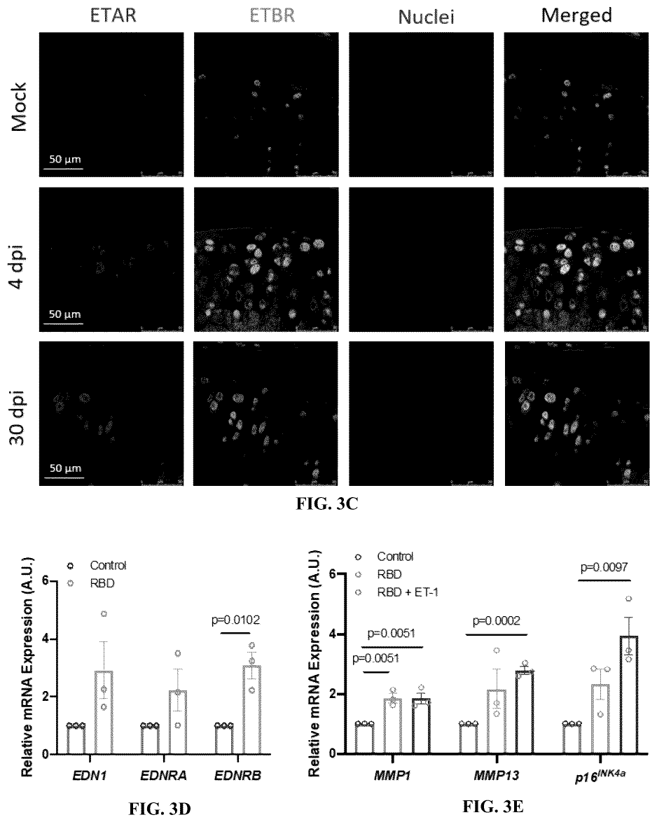

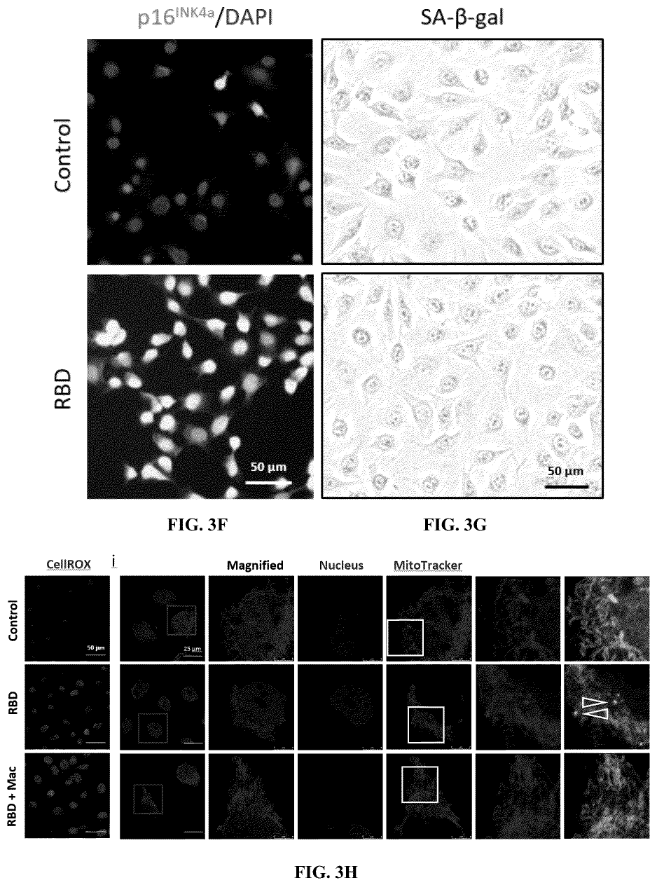

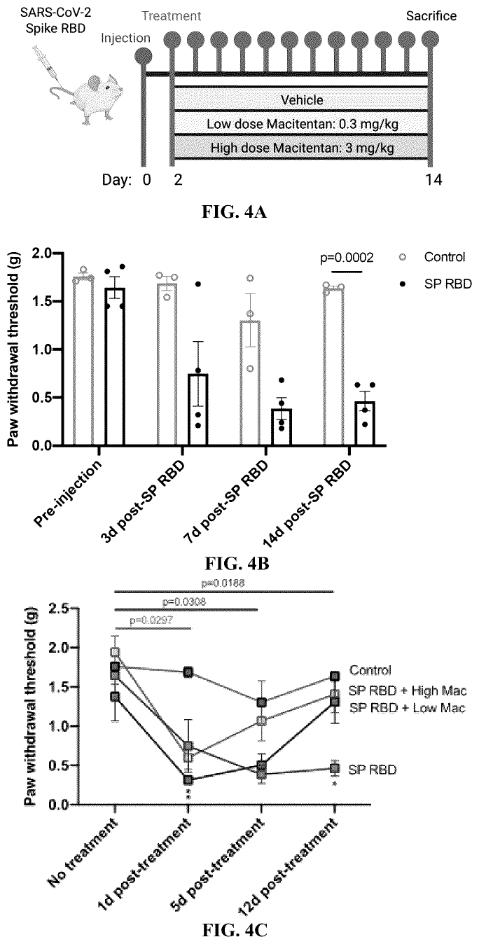

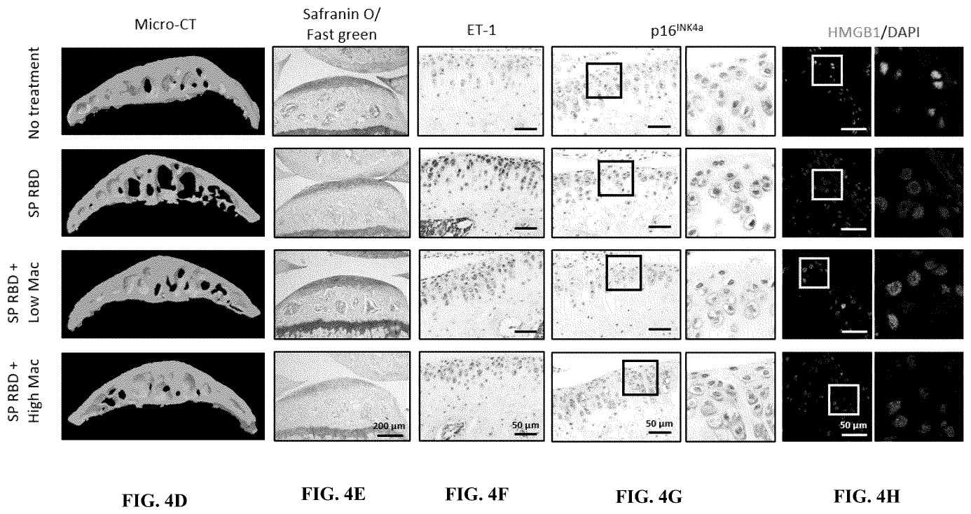

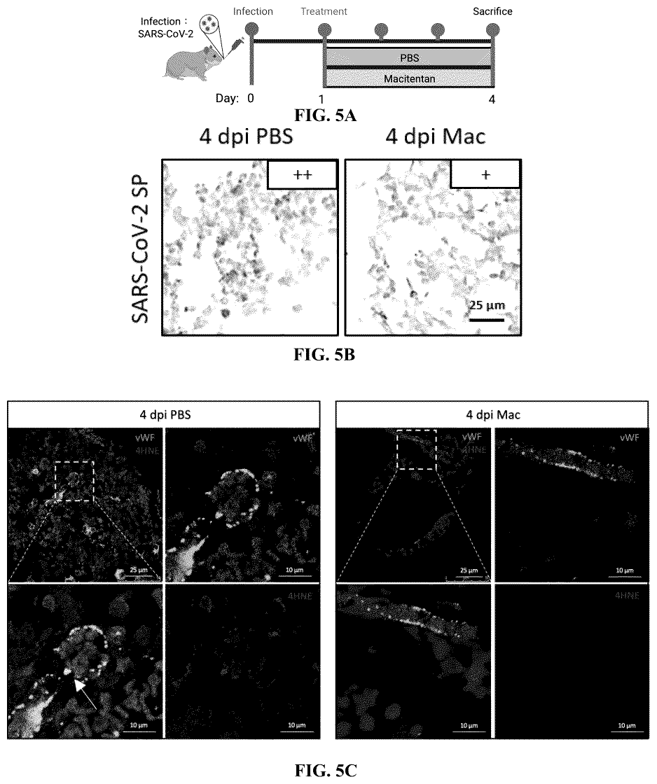

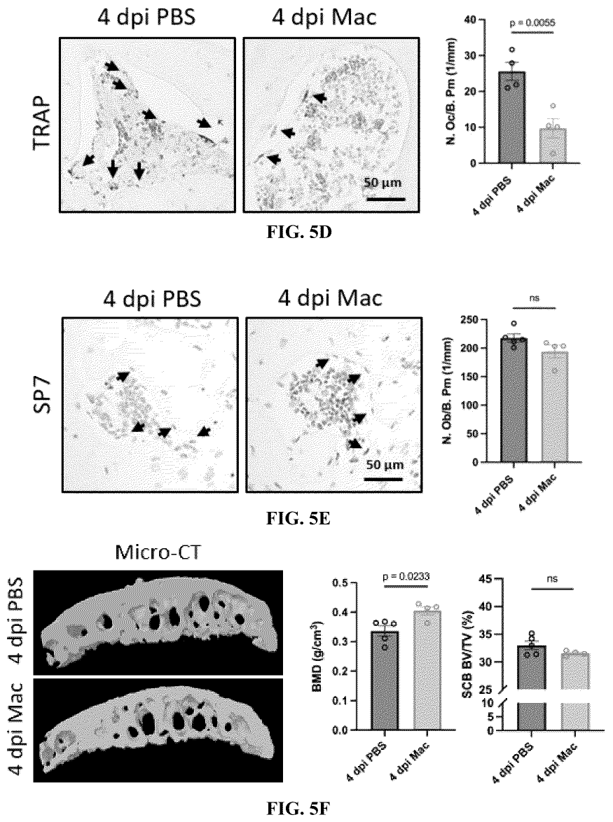

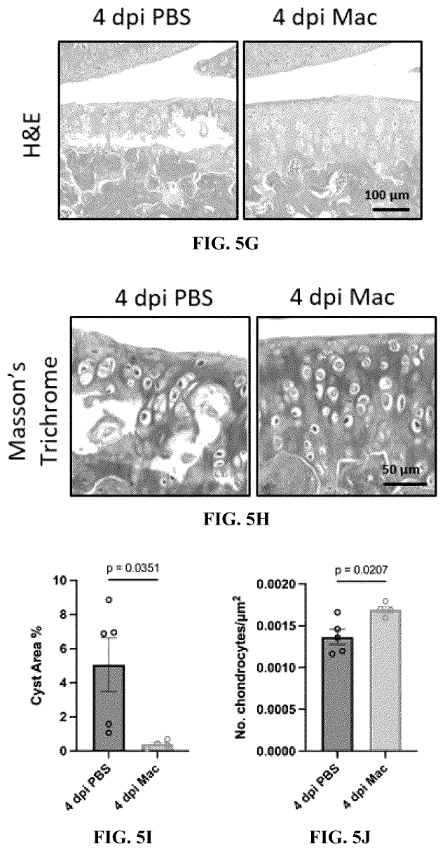

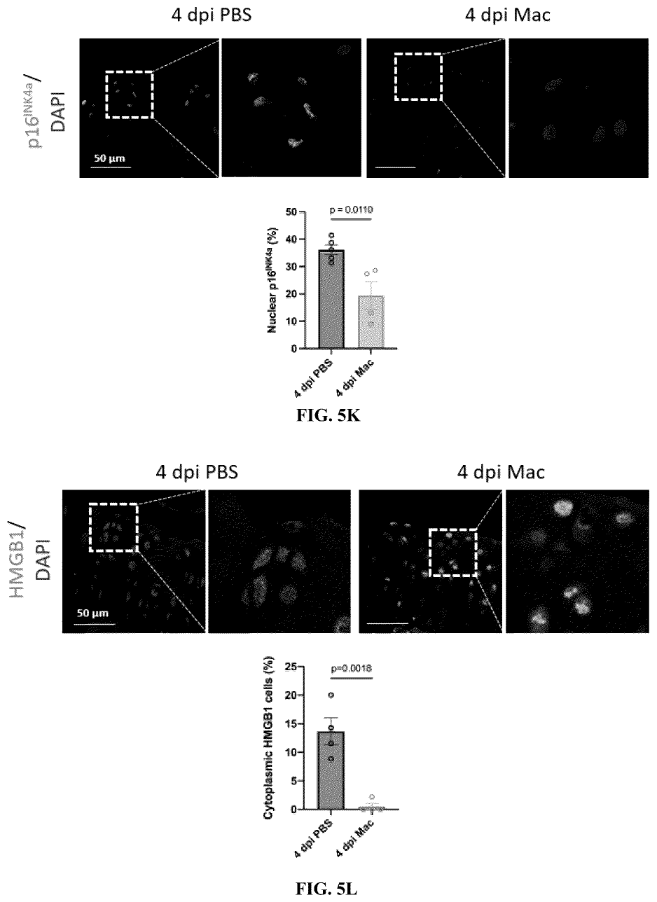

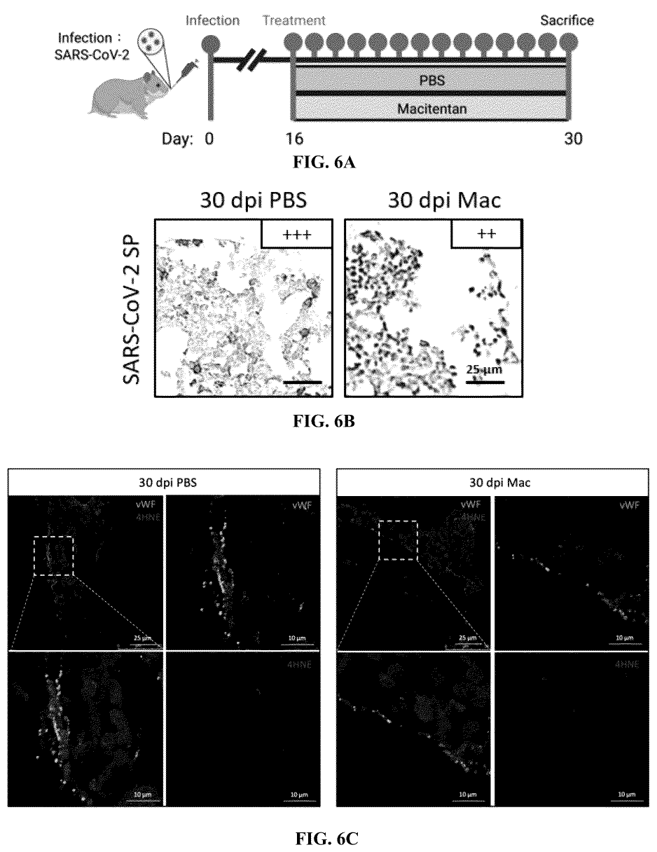

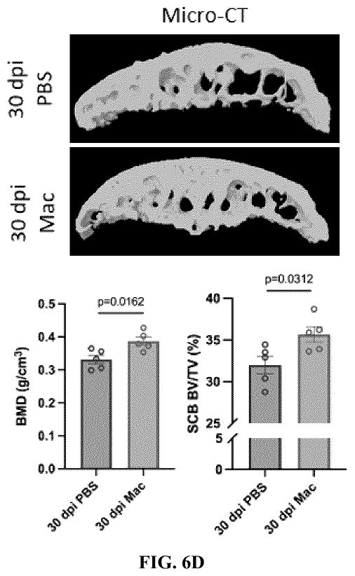

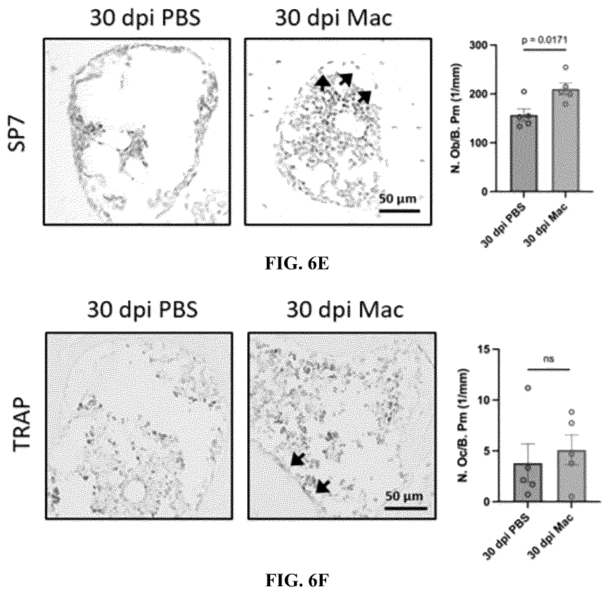

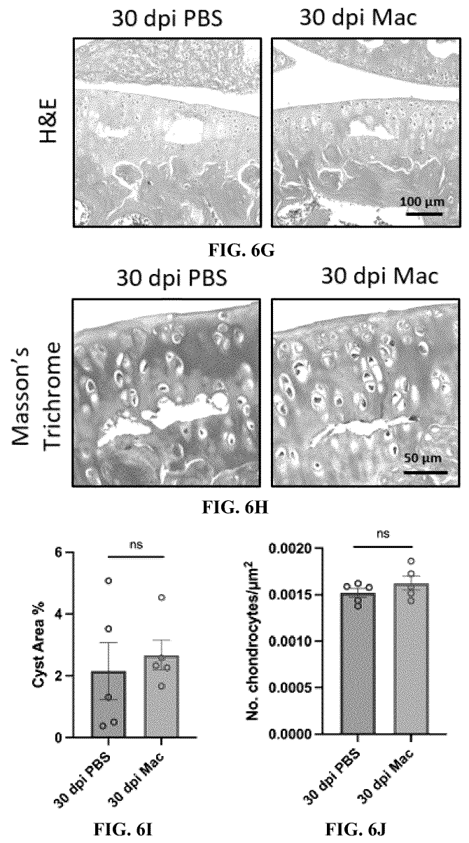

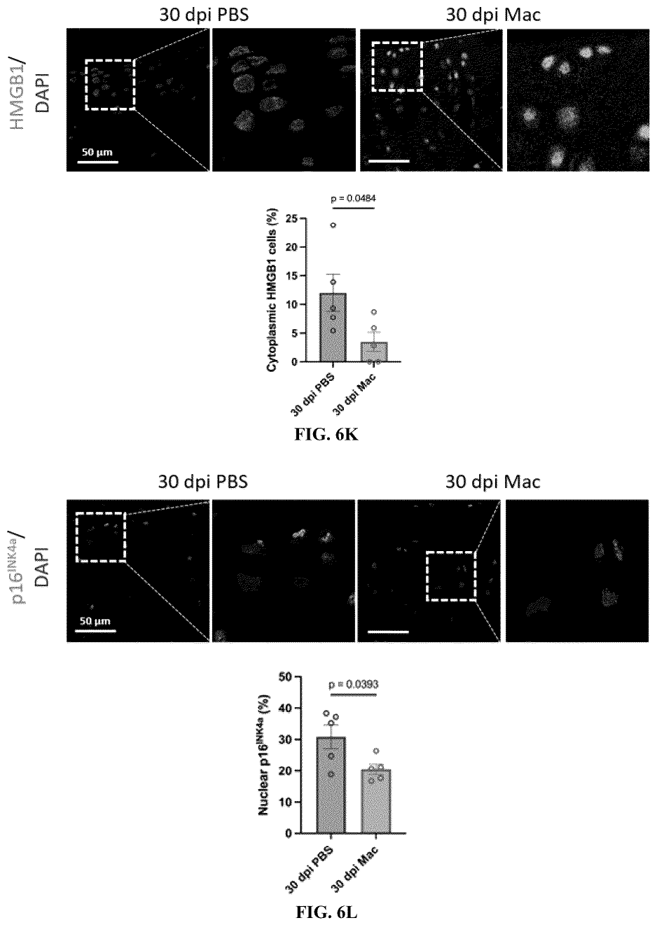

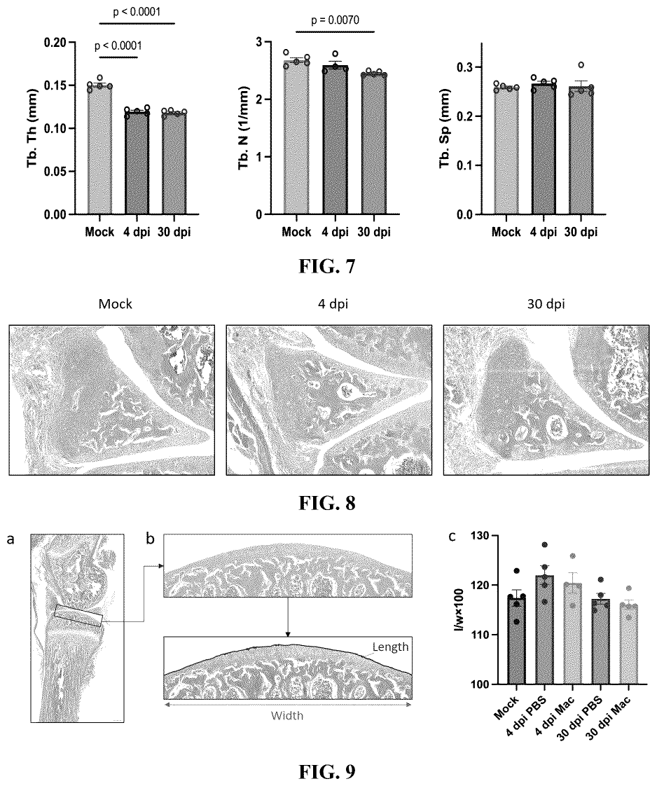

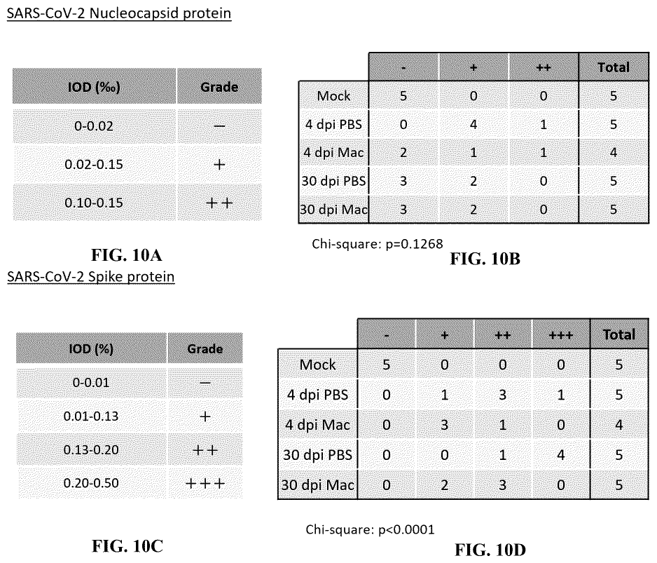

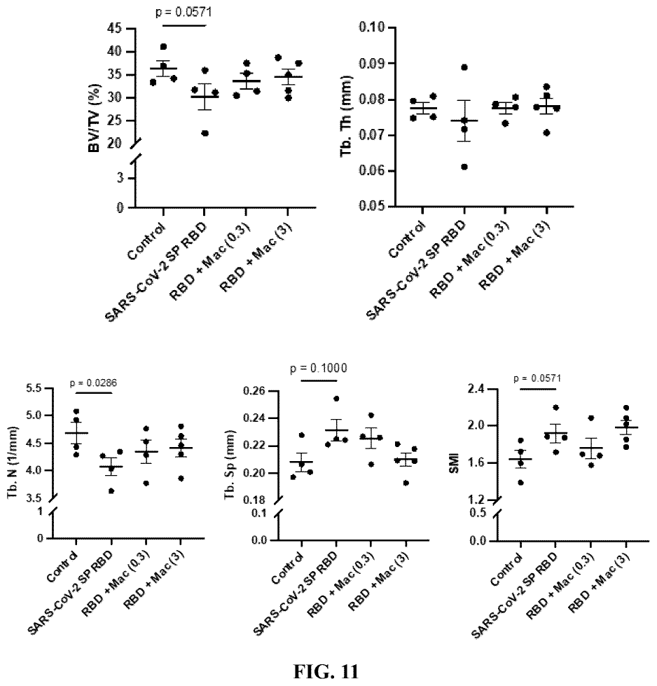

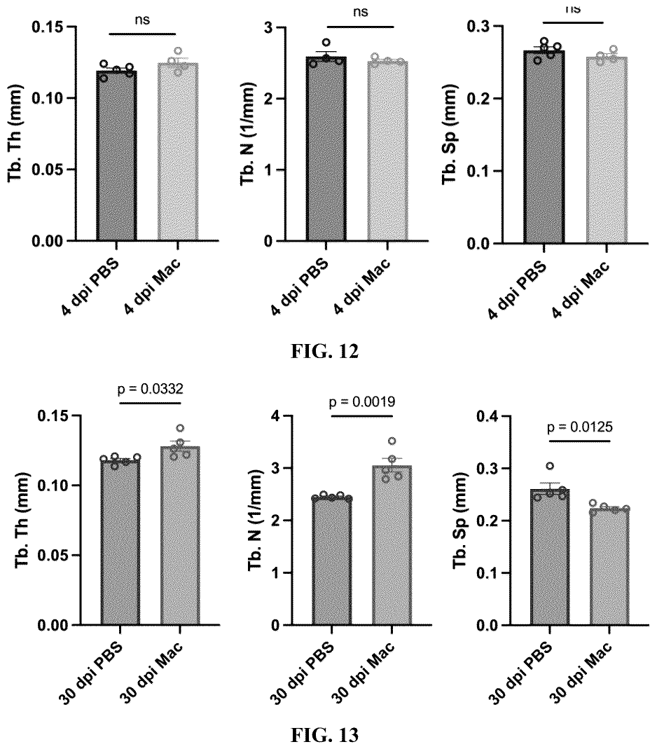

We examined structural integrity of knee joints from female golden Syrian hamsters (Mesocricetus auratus) of 10-12-week-old (FIG. 1a). At 4 days and 30 days post-infection (dpi) of SARS-COV-2, Micro-computerised tomography (μCT) assessment unravelled significantly lower subchondral bone mineral density (BMD), volume fraction (BV/TV) and trabecular thickness at both 4 and 30 dpi in the proximal epiphysis of tibia in infected hamsters compared to that of the mock infection group (FIG. 1b, 7). The number of tartrate-resistant acid phosphatase-positive (TRAP⁺) osteoclasts per bone perimeter (B. Pm) significantly increased at 4 dpi without any obvious change for the number of SP7⁺ osteoblasts per B. Pm. Noteworthily, both osteoclasts and osteoblasts dropped in sub-acute phase of infection, showing imbalanced bone remodelling at 30 dpi (FIG. 1c, d).

In addition to subchondral bone loss, we observed cyst formation at osteochondral junction in the infected animals. Cystic lesion was accompanied by a reduction of collagen content and production of fibrinous exudate as indicated by H&E and Masson's trichrome staining (FIG. 1e-g). Histopathological analysis further showed chondrocyte dropout as early as 4 dpi (FIG. 1h). Although cyst lesion tends to subside with time after infection, loss of chondrocytes and proteoglycan remained at 30 dpi (FIG. 1g-i). Senescent chondrocytes accumulated in knee joint at acute phase of SARS-COV-2 infection, i.e., 4 dpi, as indicated by nuclear translocation of p16^{INK4a} and increased cytoplasmic HMGB1 (FIG. 1j, k).

We also checked the morphology of the meniscus and synovium of the infected hamsters (FIG. 8). Roughness of articular cartilage surface was also assessed using an established protocol³² (FIG. 9). No significant change was observed in infected animals compared to the mock infection group. Taken together, SARS-COV-2 infection induces persistent osteochondral damage in hamsters.

SARS-COV-2 infection triggers endothelial dysfunction with systemic activation of endothelin signalling.

SARS-COV-2 virus and its spike protein lead to endothelial damage and dysfunction^{33, 34}, probably associated with disease manifestations in various tissues and organs. Using hydrogen peroxide as the positive control, we observed both pseudovirus expressing spike protein with D614G mutation (pV D614G) and spike protein receptor binding domain (SP RBD) of SARS-COV-2 increased cellular oxidative stress and caused mitochondrial dysfunction in endothelial cells in vitro. Pseudovirus and SP RBD stimulation resulted in change of mitochondrial dynamics from tubular to blob form (FIG. 2a, b). Upregulation of EDN1 mRNA, the protein-coding gene for the most potent vasoconstrictor, endothelin-1 (ET-1), was detected after pV or SP RBD stimulation (FIG. 2c). In addition, mRNA expression of endothelin receptors as well as receptor activator of nuclear factor kappa-B ligand (RANKL) increased, denoting enhanced bone resorption by activated osteoclasts activity.

In accordance with clinical observation in hospitalised COVID-19 patients²¹, golden Syrian hamsters infected by SARS-COV-2 showed an upregulation of serum ET-1 level as early as 4 days post-infection (FIG. 2e). Furthermore, we observed an increase of 4HNE expression, an indicator of oxidative stress, near regions with endothelial dysfunction, as shown by von Willebrand factor (vWF) staining in the subchondral bone marrow cavity (FIG. 2f), showing SARS-COV-2 affects endothelial function in the joint.

Viral spike causes chondrocyte oxidative stress and senescence with activation of endothelin signalling

Detectable viral RNA in synovial fluid post-SARS-COV-2 infection has been documented clinically³⁵. Recent post-mortem analyses also revealed detection of SARS-COV-2 nucleocapsid and spike protein in bronchial cartilage chondrocytes despite persistent negative molecular tests for SARS-COV-2 RNA, showing accumulation of viral antigens in tissues might cause deadly impact to infected patients³⁶. In line with such observation, we also found the presence of SARS-COV-2 viral proteins, i.e., nucleocapsid and spike proteins, in hamster knee joints post-infection (FIG. 3a, b, 10). Although the amount of nucleocapsid protein wore off after 30 dpi, spike proteins accumulated in the joint. Intriguingly, such viral antigens are mostly located around blood vessels in the subchondral bone marrow cavity. Viral proteins might leak from blood vessels with increased vascular permeability³⁷. This observation is also accompanied by an upregulation of endothelin receptor subtypes (ETAR and ETBR) in chondrocytes in articular cartilage post-infection compared to the mock infection group (FIG. 3c). ETBR was recently reported to transduce ET-1 signalling causing chondrocyte senescence and cartilage damage²⁴.

Hence, we treated C28/12 chondrocytes with SP RBD of SARS-COV-2 to investigate the effect of viral components on cartilage. RBD increased the gene expression of EDN1, EDNRA and EDNRB in chondrocytes (FIG. 3d). It could also upregulate matrix metalloproteinase (MMP1 and MMP13) for cartilage degradation (FIG. 3e). As indicated by qPCR and immunostaining for p16^{INK4a} and senescence-associated beta-galactosidase (SA-β-gal), RBD induced chondrocyte senescence and the synergistic effect of ET-1 and RBD drove chondrocytes to senescent state to a larger extent compared to RBD alone (FIG. 3e-g).

Endothelin receptor antagonists have been shown to lower oxidative stress and cellular senescence in a variety of in vitro models^{24, 38}. Here, we examined the efficacy of endothelin receptor antagonism on viral SP RBD-induced cellular oxidative stress and mitochondrial dynamics. Our data showed that macitentan effectively lowered SARS-COV-2 spike protein-induced increase in oxidative stress (FIG. 3h). Mitochondria changed from rod shape to blob shape after RBD challenge while macitentan showed restoration of rod-like structures (FIG. 3i). This suggests the potential therapeutic effect of macitentan to rescue SARS-COV-2-induced joint damage.

Viral spike contributes to joint pain and chondrocyte senescence To further confirm the effect of SARS-COV-2 spike protein on arthralgia and joint damage in vivo, we intravenously injected 10 μg viral spike RBD through mouse tail vein. Longitudinal monitoring of pain behaviour was performed for 2 weeks after injection of spike RBD using von Frey filament test³⁹, and the animals were sacrificed for histological evaluation (FIG. 4a). Intraperitoneal injection of macitentan at different dosages, 0.3 mg/kg and 3 mg/kg, was also assessed for joint pain relief starting from two days after RBD injection.

Paw withdrawal threshold in response to mechanical stimuli reduced from three days post-SP RBD injection and lasted for at least 2 weeks, indicating hypersensitivity occurs at both acute and sub-acute phase post-injection (FIG. 4b). Macitentan at high dose started showing beneficial effect in pain sensitisation reflected by an increase in paw withdrawal threshold after 5 days of treatment. Low-dose macitentan was also able to attain the same nociceptive tolerance as high dose after 12 days of the treatment regimen (FIG. 4c).

We further investigated the structural and cellular changes in articular joints after RBD challenge as well as the treatment effect of macitentan. Similar to hamsters infected by SARS-COV-2, viral spike RBD could induce subchondral bone loss in mice injected with spike protein RBD (FIG. 4d, 10). Loss of proteoglycan and surface fibrillation were observed in articular cartilage in RBD-treated mice (FIG. 4e). RBD also caused upregulation of ET-1, nuclear p16^{INK4a} translocation and cytoplasmic HMGB1 translocation at articular cartilage (FIG. 4f-h), showing spike protein RBD alone leads to chondrocyte senescence in a mouse model. Macitentan at 0.3 mg/kg performed better in terms of preserving proteoglycan and preventing accumulation of senescent chondrocytes despite the faster analgesic effect at the higher dose (FIG. 4e-h). Therefore, we chose the low dose of macitentan for further investigation.

Early macitentan treatment partially rescues SARS-COV-2-induced osteochondral damage To study the efficacy of macitentan on authentic SARS-COV-2 infection, macitentan was injected intraperitoneally daily at a dose of 0.3 mg per kg body weight to infected hamsters one day after virus exposure. The treatment dosage was chosen based on our trial using viral spike RBD mouse model (FIG. 4). The animals were euthanised at 4 dpi to study the early treatment effect on acute joint damage (FIG. 5a). Macitentan tended to lower the amount of viral spike protein at local joint tissue (FIG. 5b). The early treatment also lowered vWF and 4HNE expression level in the bone marrow cavity of subchondral bone, showing macitentan improved vascular function as well as lowered oxidative stress (FIG. 5c). Compared with vehicle (PBS) group, animals treated with macitentan showed a significantly lower number of TRAP⁺ osteoclasts in subchondral bone without affecting the number of SP7⁺ osteoblasts progenitors (FIG. 5d, e). As a result, short-term macitentan treatment at acute phase could restore subchondral BMD although there was no significant difference in terms of BV/TV at 4 dpi, possibly due to short treatment duration, i.e., 4-day (FIG. 5f, 12).

More intriguingly, macitentan treatment mitigated SARS-COV-2-induced cartilage damage by effectively reducing the area of the cysts and decreasing the fibrin exudate at the osteochondral junction (FIG. 5g-i). It also prevented virus-induced collagen loss as shown by Masson's trichrome staining (FIG. 5h). Number of chondrocytes is a key factor for cartilage homeostasis and disease, indicating the capability of matrix synthesis and replenishment⁴⁰. More chondrocytes were observed in articular cartilage in hamsters treated with macitentan compared to the vehicle-treated group (FIG. 5j). We also found that early macitentan treatment effectively prevented the accumulation of senescent chondrocytes, reflected by a decrease in the number of nuclear p16^{INK4a} and cytoplasmic HMGB1 cells in articular cartilage (FIG. 5k, l). Collectively, early macitentan treatment could effectively alleviate SARS-COV-2-induced acute joint damage.

Delayed macitentan treatment alleviates subchondral bone loss at sub-acute phase of SARS-COV-2 infection

Due to the limited healing capacity of articular cartilage, SARS-COV-2-induced osteochondral damage sustained till 30 dpi in infected hamsters (FIG. 1). It would help understand the possible cause of underlying fluctuant joint pain up to weeks or months in clinical reports41,42. Therefore, it is worthwhile to explore whether delayed macitentan treatment after acute phase of SARS-COV-2 infection remains effective to rescue osteochondral damage.

Macitentan treatment was initiated at 16 dpi and lasted for 2 weeks till 30 dpi, which is regarded as sub-acute phase of SARS-COV-2 infection (FIG. 6a). We found that delayed macitentan treatment could reduce the amount of viral spike protein retained and restore virus-induced structural damage to the joint (FIG. 6b). Oxidative stress in subchondral bone subsided at 30 dpi as indicated by 4HNE immunostaining. However, vWF, a marker for endothelial dysfunction, was still present in bone marrow yet at a lower level compared to acute phase. Macitentan treatment at sub-acute phase could effectively lower the expression level of vWF. It indicated that delayed treatment might also improve vascular function in the joint (FIG. 6c).

Micro-CT analysis showed an improvement in subchondral bone mass and microstructure in tibial epiphysis including BMD, BV/TV, trabecular thickness, number and separation after macitentan treatment on infected animals (FIGS. 6d, 13). In contrast to the acute phase, treatment at sub-acute phase of viral infection increased the number of SP7⁺ osteoblast progenitors without affecting the number of TRAP⁺ osteoclasts (FIG. 6e, f). The slight yet not significant improvement in terms of cyst formation and chondrocyte dropout after one month of infection was likely due to active growth of the hamsters under puberty. However, due to the incapability of self-repair in articular cartilage, delayed macitentan treatment at sub-acute phase did not lead to further improvement in articular cartilage structurally (FIG. 6g-j). Nevertheless, delayed treatment could effectively lower the number of senescent chondrocytes in articular cartilage as indicated by p16^{INK4a} and HMGB1 staining (FIG. 6k, l). In short, delayed macitentan treatment can still alleviate virus-induced osteochondral damage.

### METHODS

### SARS-COV-2 Virus and Biosafety

SARS-COV-2 (HKU-001a strain, GenBank accession number: MT230904) was isolated from the nasopharyngeal aspirate specimen of a COVID-19 patient in Hong Kong⁶¹. The virus was amplified in VeroE6 cells to prepare working stock as previously described⁶². All experiments involving the use of true SARS-COV-2 virus followed the standard operating procedures of the Biosafety Level 3 facility at the University of Hong Kong (HKU).

### Golden Syrian Hamster Infection Model for SARS-COV-2

All the procedures were approved by HKU Committee on the Use of Live Animals in Teaching and Research (CULATR). Female golden Syrian hamsters (Mesocricetus auratus) of 10-12-week-old were infected intranasally at 0 day post-infection (dpi) with 10⁵ PFU of SARS-COV-2 in 50 μl PBS under general anaesthesia using ketamine (200 mg/kg)-xylazine (10 mg/kg) cocktail in the Biosafety Level 3 animal facility at HKU Department of Microbiology according to our established protocol^{63, 64}. Fifty-microliter of PBS was used in mock-infected hamsters. Hamsters were sacrificed at 0, 4 or 30 dpi, serum and hind limbs were collected for subsequent analyses.

### Mouse Model with SARS-COV-2 Spike Protein RBD Treatment

Animal Subjects Ethics Sub-Committee (ASESC) of the Hong Kong Polytechnic University approved all the experimental procedures (22-23/363-BME-R-GRF). Female Balb/c mice of 6-month-old were used in the study. Briefly, the mice were anaesthetised using isoflurane (3-4% for induction and 1-2% for maintenance). Ten μg of recombinant wildtype SARS-COV-2 spike protein receptor binding domain (RBD) was injected intravenously through tail vein. Same volume of PBS was injected in control group. Animals were sacrificed 14 days post-injection. Hind limbs were collected for micro-CT and histological analyses.

### In Vivo Macitentan Treatment

For mouse model with RBD, we used 2 different dosages of Macitentan (Actelion Pharmaceuticals Ltd, Allschwil, Switzerland) (0.3 mg/kg and 3 mg/kg). Intraperitoneal injection was performed daily starting from 2 days after RBD injection. For hamster infection model with authentic SARS-COV-2 virus, 0.3 mg/kg Macitentan was used. The dosage is equivalent to 2.34 mg per day for a 60 kg person⁶⁵. Two treatment regimens were employed to study the different treatment timeframes. For early treatment, Macitentan was intraperitoneally administered at 1 dpi and lasted for 4 days. For delayed treatment, the drug was only given the same way at 16 dpi and lasted for 2 weeks. Animals were sacrificed at the end of treatment.

### Von Frey Filament Test

Nociceptive tolerance of mice was assessed by using an electronic von Frey aesthesiometer (IITC, USA) with a detection range from 0 to 74 g according to a previously published simplified up-down method³. Training for the animals was performed for 3 days before the formal measurement. Briefly, the animal was placed on an elevated mesh grid where it stayed for at least 15 minutes to adapt to the environment. The whole setting was carried out in a black box to keep the animal calm. Briefly, middle filament #3 was selected as the starting filament after trials.

Filaments were applied to the mid-plantar surface of the hind paw and withdrawal response was recorded. The next filament was chosen based on the response of the mouse. The exact force was recorded after 5 presentations with an adjustment factor of ±0.5. Von Frey filament test was performed before, 3-, 7-and 14-day after injection of spike protein RBD. The improvement of nociceptive tolerance was evaluated by comparing the readings between RBD and Macitentan-treated groups.

### Measurement of Serum ET-1 Level by ELISA

Blood was collected from the hamsters when sacrificed and rested at room temperature for 20 minutes before centrifugation at 1500×g for 10 minutes at 4° C. to get sera. Endothelin-1 ELISA kit (ab133030, Abcam) was used to quantify ET-1 in sera according to the manufacturer's protocol. Heat-inactivated serum samples were added to antibody-coated plates for the assay. The absorbance was measured at 450 nm and the concentration was determined.

### Microcomputed Tomography (μCT) Analysis

Bone samples from hamsters and mice were fixed in 4% paraformaldehyde for 48 h followed by 24 h in 70% ethanol to inactivate the pathogens in a biosafety level-3 facility when necessary. The specimens were then scanned using a high-resolution micro-CT system (1276, SkyScan, Kontich, Belgium) at a resolution of 13.0 μm per pixel. 90 kV voltage and 200μA current were used during the scans. For calibration of bone mineral density (BMD), phantoms with standard densities of 0.25 and 0.75 g/cm³ were used. 3D reconstruction and analysis were done using NRecon and CTAn software (Skyscan Company) respectively. One-hundred images covering the trabecular bone in proximal epiphysis above growth plate in tibia were selected as the region of interest (ROI) for measurement of trabecular bone parameters, including bone volume fraction (BV/TV) and bone mineral density (BMD), trabecular bone number (Tb. N.), thickness (Tb. Th.), and separation (Tb. Sp.). Three-dimensional structures were generated using CTvol (Skyscan Company) using 20 images in the middle of the joint. Measurement of mice specimens was the same except resolution of 8.67 μm per pixel was used. 48 kV voltage and 179-μA current were used during the scans.

### Histological Analysis

Bone samples from hamsters and mice were harvested and fixed. Tibias were decalcified using 10% ethylenediaminetetraacetic acid (EDTA) at pH 7.4. The specimens were dehydrated with ethanol and infiltrated with xylene. The samples were then embedded in paraffin. Five-micron sections were made for histological analyses. Sections were stained with haematoxylin and eosin (H&E) (ab245880, Abcam), safranin O/fast green (Alfa Aesar), Masson's trichrome (Abcam) and tartrate-resistant acid phosphatase (TRAP) (Amizona Scientific, US) according to the standard staining protocols for histopathological evaluation. Roughness of articular cartilage was determined following a published protocol³². The severity of cyst lesion was quantified by the percentage of cyst area divided by total area of articular cartilage.

Immunohistochemistry and immunocytochemistry Sections were stained with different antibodies to detect specific proteins in our samples. Briefly, antigen retrieval was done using either citrate buffer or Tris/ETDA following the suggestion on the product sheets. Quenching of endogenous peroxidase activity was performed using hydrogen peroxide. The sections were then incubated with primary antibodies overnight at 4□ C after blocking. Antibodies used: Anti-SARS-COV-2 spike protein RBD (MA5-36247), Anti-SARS-COV-2 nucleocapsid protein (MA17404, Invitrogen), Anti-endothelin-1 (ab117757, Abcam), Anti-ETAR (ab117521, Abcam), Anti-ETBR (ab117529, Abcam), Anti-p16^{INK4a} (ab189034 &ab54210, Abcam) and Anti-HMGB1 (ab18256, Abcam), Anti-SP7 (ab209484, Abcam) and Anti-Caspase 3 (43-7800, Invitrogen). Visualisation of positive signal was done with either diaminobenzidine (DAB) staining (Vector Lab, US) or fluorescent secondary antibodies (Invitrogen). Semi-quantification of NP and SP was done by measuring integrated optical density (IOD) of DAB staining. Exact grading method was shown in FIG. 10.

For multichannel staining for ETAR and ETBR, DendronFluor®multi-channel IHC detection kit (Neon, Histova, China) was used. Primary antibody was incubated at 4□ C overnight and tyramide signal amplification (TSA) was performed the next day, followed by a new antibody each day. Visualisation of positive signals was done with horseradish peroxidase (HRP) secondary antibody and different fluorophores: NEON520 and NEON570. SN520 was used as a nuclear stain. OLYMPUS BX51WI and Leica TCS SPE confocal microscope were used to capture the images.

For immunocytochemistry on chondrocytes, cells grown on coverslips were incubated with Anti-p16^{INK4a} (ab 189034 , Abcam) at 4□ C overnight after fixation, permeabilization and blocking. A secondary antibody was applied the next day for visualisation of positive signals, Images were captured using Leica TCS SPE confocal microscope. All the image analyses were performed using Image-Pro Plus 6.0 and ImageJ.

### Preparation of Pseudovirus and Recombinant SARS-COV-2 S1 Receptor Binding Domain (RBD)

Pseudovirus-bearing D614G spike protein was produced using Lenti-X lentiviral packaging plasmid (632673, Takara) according to the manufacturer's protocol. In brief, the plasmid was first amplified and diluted before mixing with the transfection reagent. The resultant DNA-packaging mix complex was then added to 293T cells. The supernatant was then harvested and the virus titre was determined. HUVEC, C28/12 and ATDC cells were infected with pseudovirus (MOI of 10) and collected for analysis after 24 hours of incubation. The recombinant receptor binding domain (RBD) (residue 306-543) of SARS-COV-2 spike protein was synthesised from the reference sequence of Wuhan-Hu-1 (GenBank ID YP_009724390.1). It was expressed and purified in E. coli with modifications according to the previous publication 66.

### Cell Culture

Chondrocyte cell lines C28/12 (human), ATDC5 (mouse) and human umbilical vein endothelial cells (HUVEC) were received as gifts from Prof. Danny Chan from the University of Hong Kong, Prof. Limin Rong from Sun Yat-Sen University and Prof.

Mo Yang from the Hong Kong Polytechnic University respectively. Both chondrocyte cell lines were maintained in DMEM/F12 culture medium supplemented with 5% heat-inactivated FBS, 50 U ml⁻¹ penicillin and 50 μg ml-¹ streptomycin. HUVEC were cultured in endothelial cell medium with 1% endothelial cell growth supplement and 1% antibiotic solution (ScienCell).

### In Vitro Cell Model

To study the direct effect of spike protein RBD and endothelin-1 (ET-1) on chondrocytes, ATDC5, C28/12 or HUVEC cells were treated with 10 μg/ml RBD or pV D614G (MOI of 10) or/and 100 nM ET-1 for 24 h. Cell lysates were collected for subsequent RNA extraction and real-time PCR for gene expression study. Staining for senescence marker p16^{Ink4a} and beta-galactosidase (β-gal) were performed.

To investigate the rescue effect of Macitentan on RBD-induced oxidative stress accumulation and change in mitochondrial dynamics, cells seeded on coverslips were pre-treated with 10 μM Macitentan for 30 minutes before adding RBD for an hour.

### Senescence β-Galactosidase Activity Assay

β-galactosidase (β-gal) staining was performed on chondrocytes using a senescence-associated-β-galactosidase (SA-β-gal) staining kit (CST) according to the manufacturer's instructions. In brief, the cells were washed in PBS and fixed, followed by staining with β-Galactosidase staining solution at 37° C. in a dry incubator overnight. Nikon Eclipse TS100 was used to capture the images.

### Evaluation of Cellular Oxidative Stress and Mitochondrial Morphology

To evaluate cellular oxidative stress, CellROX® Deep Red Reagent solution (Invitrogen) was used according to the manufacturer's instructions. Positive signal was visualized by excitation/emission at 640/665 nm. For visualising mitochondrial morphology, we stained the cells using Mito Tracker® Red CMXRos (Invitrogen) with excitation/emission at 579/599 nm. Cells were then counterstained and mounted by ProLong® Gold antifade reagent with 4′, 6-diamidino-2-phenylindole (DAPI). All the images were captured using Leica TCS SPE Confocal microscope.

### Real-Time Quantitative Polymerase Chain Reaction (RT-qPCR) Assay of Cultured Cells

Total RNA was extracted from the cultured cells using E.Z.N.A.®TOTAL RNA KIT (Omega) according to the manufacturer's instructions. RNA was quantified and reversed transcribed into cDNA using RevertAid First Strand cDNA Synthesis Kit (Thermofisher). RT-PCR was then performed by setting up the reaction mix using QuantiNova SYBR Green PCR Kit (Qiagen) and specific primers (Supplementary Table 1), then run in BIORAD CFX96 Touch™ Real-Time PCR Detection System. Expression of GAPDH was used to normalize the expression levels of other genes.

### Statistical Analysis

All data were presented as mean±S.E.M. unless specified. The exact sample size per group was shown in the figure legends. Each data point was represented by a dot on each graph. Comparisons among time course differences (0, 4, 30 dpi) were performed using one-way ANOVA or Kruskal-Wallis test when deemed appropriate.

For comparison of the infected and treatment group at different time points, unpaired two-tailed Student's t-test or Mann-Whitney test was carried out depending on the distribution of the data set. Respective post-hoc tests such as Tukey's multiple-comparison post hoc test or Bonferroni's post hoc test were carried out when overall significance was detected between groups. Two-way ANOVA with Tukey post hoc correction for multiple testing was used for comparing the paw withdrawal threshold of different groups at different time points. The level of significance was set at p<0.05. Analyses and graphs were generated using Prism 8 (GraphPad).

### REFERENCE

1 Group, P.-C.C. Clinical characteristics with inflammation profiling of long COVID and association with 1-year recovery following hospitalisation in the UK: a prospective observational study. Lancet Respir Med 10, 761-775, doi: 10.1016/S2213-2600 (22) 00127-8 (2022).
2 Qiao, W. et al. SARS-COV-2 infection induces inflammatory bone loss in golden Syrian hamsters. Nat Commun 13, 2539, doi: 10.1038/s41467-022-30195-w (2022).
3 Nalbandian, A. et al. Post-acute COVID-19 syndrome. Nat Med 27, 601-615, doi: 10.1038/s41591-021-01283-z (2021).
4 Heesakkers, H. et al. Clinical Outcomes Among Patients With 1-Year Survival Following Intensive Care Unit Treatment for COVID-19. JAMA 327, 559-565, doi: 10.1001/jama. 2022.0040 (2022).
5 Carfi, A., Bernabei, R., Landi, F. &Gemelli Against, C.-P.-A. C.S.G. Persistent Symptoms in Patients After Acute COVID-19. JAMA 324, 603-605, doi: 10.1001/jama. 2020.12603 (2020).
6 Chaudhry, Z. S., Nellessen, N., Reis, C. & Sharip, A. The development of inflammatory arthritis following SARS-COV-2 infection: a systematic review of the literature. Fam Pract, doi: 10.1093/fampra/cmac029 (2022).
7 Fernandez-de-Las-Penas, C. et al. Prevalence and risk factors of musculoskeletal pain symptoms as long-term post-COVID sequelae in hospitalized COVID-19 survivors: a multicenter study. Pain 163, e989-e996, doi: 10.1097/j. pain. 0000000000002564(2022 ).
8 Fernandez-de-Las-Penas, C., Navarro-Santana, M., Plaza-Manzano, G., Palacios-Cena, D. &Arendt-Nielsen, L. Time course prevalence of post-COVID pain symptoms of musculoskeletal origin in patients who had survived severe acute respiratory syndrome coronavirus 2 infection: a systematic review and meta-analysis. Pain 163, 1220-1231, doi: 10.1097/j. pain. 0000000000002496 (2022).
9 Siva, C., Velazquez, C., Mody, A. & Brasington, R. Diagnosing acute monoarthritis in adults: a practical approach for the family physician. Am Fam Physician 68, 83-90 (2003).
10 Joo, Y. B., Lim, Y. H., Kim, K. J., Park, K. S. & Park, Y. J. Respiratory viral infections and the risk of rheumatoid arthritis. Arthritis Res Ther 21, 199, doi: 10.1186/s13075-019-1977-9 (2019).
11 Hoong, C. W. S., Amin, M., Tan, T. C. & Lee, J. E. Viral arthralgia a new manifestation of COVID-19 infection? A cohort study of COVID-19-associated musculoskeletal symptoms. Int J Infect Dis 104, 363-369, doi: 10.1016/j. ijid. 2021.01.031(2021 ).
12 Slouma, M. et al. Reactive arthritis occurring after COVID- 19 infection: a narrative review. Infection, doi: 10.1007/s15010-022-01858-z (2022).
13 Honge, B. L., Hermansen, M. F. & Storgaard, M. Reactive arthritis after COVID-19. BMJ Case Rep 14, doi: 10.1136/bcr-2020-241375 (2012).
14 Kocyigit, B. F. &Akyol, A. Reactive arthritis after COVID-19: a case-based review. Rheumatol Int 41, 2031-2039, doi: 10.1007/s00296-021-04998-x (2021).
15 Ono, K. et al. Reactive arthritis after COVID-19 infection. RMD Open 6, doi:
10.1136/rmdopen-2020-001350 (2020).
16 Taha, S. I., Samaan, S. F., Ibrahim, R. A., El-Sehsah, E. M. & Youssef, M .K. Post-COVID-19 arthritis: is it hyperinflammation or autoimmunity? Eur Cytokine Netw 32,83-88, doi: 10.1684/ecn. 2021.0471(2021).
17 Son, K. et al. Circulating anti-nuclear autoantibodies in COVID-19 survivors predict long-COVID symptoms. Eur Respir J, doi: 10.1183/13993003.00970-2022 (2022).
18 Mendez, R. et al. Acute and sustained increase in endothelial biomarkers in COVID-19. Thorax 77, 400-403, doi: 10.1136/thoraxjnl-2020-216797 (2022).
19 Lampsas, S. et al. The Role of Endothelial Related Circulating Biomarkers in COVID-19. A Systematic Review and Meta-analysis. Curr Med Chem 29, 3790-3805, doi: 10.2174/0929867328666211026124033 (2022).
20 Willems, L. H. et al. Sustained inflammation, coagulation activation and elevated endothelin- 1 levels without macrovascular dysfunction at 3 months after COVID-19. Thromb Res 209, 106-114, doi: 10.1016/j. thromres. 2021.11.027(2022 ).
21 Abraham, G. R. et al. Endothelin-1 is increased in the plasma of patients hospitalised with Covid- 19. J Mol Cell Cardiol 167, 92-96, doi: 10.1016/j. yjmcc. 2022.03.007(2022 ).
22 Atar, M. O., Ozcakar, L., Gencturk, Z. & Aytur, Y. Serum endothelin-1 levels, radiographic and ultrasonographic evaluations, and clinical parameters in patients with knee and/or hand osteoarthritis. J Back Musculoskelet Rehabil 32, 549-554, doi: 10.3233/BMR-181326 (2019).
23 Zhao, Z., Li, E., Cao, Q., Sun, J. & Ma, B. Endothelin-1 concentrations are correlated with the severity of knee osteoarthritis. J Investig Med 64, 872-874, doi: 10.1136/jim-2015-000030 (2016).
24 Au, M., Liu, Z., Rong, L., Zheng, Y. &Wen, C. Endothelin-1 induces chondrocyte senescence and cartilage damage via endothelin receptor type B in a post-traumatic osteoarthritis mouse model. Osteoarthritis and cartilage/OARS, Osteoarthritis Research Society, doi: 10.1016/j. joca. 2020.08.006(2020 ).
25 De-Melo, J. D., Tonussi, C. R., D'Orleans-Juste, P. & Rae, G. A. Effects of endothelin-1 on inflammatory incapacitation of the rat knee joint. J Cardiovasc Pharmacol 31 Suppl 1, S518-520, doi: 10.1097/00005344-199800001-00149 (1998).
26 De-Melo, J. D., Tonussi, C. R., D'Orleans-Juste, P. & Rae, G. A. Articular nociception induced by endothelin-1, carrageenan and LPS in naive and previously inflamed knee-joints in the rat: inhibition by endothelin receptor antagonists. Pain 77, 261-269, doi: 10.1016/S0304-3959 (98) 00098-0 (1998).
27 Roy-Beaudry, M. et al. Endothelin 1 promotes osteoarthritic cartilage degradation via matrix metalloprotease 1 and matrix metalloprotease 13 induction. Arthritis Rheum 48, 2855-2864, doi: 10.1002/art. 11247(2003 ).
28 Kaufman, G. N., Zaouter, C., Valteau, B., Sirois, P. & Moldovan, F. Nociceptive tolerance is improved by bradykinin receptor B1 antagonism and joint morphology is protected by both endothelin type A and bradykinin receptor B1 antagonism in a surgical model of osteoarthritis. Arthritis Res Ther 13, R76, doi: 10.1186/ar3338 (2011).
29 Imhof, A. K. et al. Potent anti-inflammatory and antinociceptive activity of the endothelin receptor antagonist bosentan in monoarthritic mice. Arthritis Res Ther 13, R97, doi: 10.1186/ar3372 (2011).
30 Khodorova, A., Montmayeur, J. P. & Strichartz, G. Endothelin receptors and pain. J Pain 10, 4-28, doi: 10.1016/j. jpain. 2008.09.009(2009 ).
31 Chan, J. F. et al. Simulation of the Clinical and Pathological Manifestations of Coronavirus Disease 2019 (COVID-19) in a Golden Syrian Hamster Model: Implications for Disease Pathogenesis and Transmissibility. Clin Infect Dis 71, 2428-2446, doi: 10.1093/cid/ciaa325 (2020).
32 Pastoureau, P., Leduc, S., Chomel, A. & De Ceuninck, F. Quantitative assessment of articular cartilage and subchondral bone histology in the meniscectomized guinea pig model of osteoarthritis. Osteoarthritis and cartilage/OARS, Osteoarthritis Research Society 11, 412-423, doi: 10.1016/s1063-4584 (03) 00050-5 (2003).
33 Lei, Y. et al. SARS-COV-2 Spike Protein Impairs Endothelial Function via Downregulation of ACE 2. Circ Res 128, 1323-1326, doi: 10.1161/CIRCRESAHA. 121.318902(2021 ).
34 Dupont, A. et al. Vascular Endothelial Damage in the Pathogenesis of Organ Injury in Severe COVID-19. Arterioscler Thromb Vasc Biol 41, 1760-1773, doi: 10.1161/ATVBAHA. 120.315595 (2021).
35 Kuschner, Z., Ortega, A. & Mukherji, P. A case of SARS-COV-2-associated arthritis with detection of viral RNA in synovial fluid. J Am Coll Emerg Physicians Open 2, e12452, doi: 10.1002/emp2.12452 (2021).
36 Bussani, R. et al. Persistent SARS-COV-2 infection in patients seemingly recovered from COVID- 19. J Pathol 259, 254-263, doi: 10.1002/path. 6035(2023 ).
37 Rauti, R. et al. Effect of SARS-COV-2 proteins on vascular permeability. Elife 10, doi: 10.7554/eLife. 69314 (2021).
38 Alcalde-Estevez, E. et al. Endothelin-1 induces cellular senescence and fibrosis in cultured myoblasts. A potential mechanism of aging-related sarcopenia. Aging (Albany NY) 12, 11200-11223, doi: 10.18632/aging. 103450 (2020).
39 Robert P. Bonin, Bories, C. & Koninck, Y. D. A simplified up-down method (SUDO) for measuring mechanical nociception in rodents using von Frey filaments. Molecualr Pain (2014).
40 Bobacz, K., Erlacher, L., Smolen, J., Soleiman, A. & Graninger, W. B.

Chondrocyte number and proteoglycan synthesis in the aging and osteoarthritic human articular cartilage. Ann Rheum Dis 63, 1618-1622, doi: 10.1136/ard. 2002.002162 (2004).
41 Zhang, H. et al. Data-driven identification of post-acute SARS-COV-2 infection subphenotypes. Nat Med, doi: 10.1038/s41591-022-02116-3 (2022).
42 Herndon, C. M. & Nguyen, V. Patterns of Viral Arthropathy and Myalgia Following COVID-19: A Cross-Sectional National Survey. J Pain Res 15, 3069-3077, doi: 10.2147/JPR. S373295(2022 ).
43 Lopez-Leon, S. et al. More than 50 Long-term effects of COVID-19: a systematic review and meta-analysis. medRxiv, doi: 10.1101/2021.01.27.21250617 (2021).
44 Drevensek, M., Sprogar, S., Boras, I. & Drevensek, G. Effects of endothelin antagonist tezosentan on orthodontic tooth movement in rats. Am J Orthod Dentofacial Orthop 129, 555-558, doi: 10.1016/j. ajodo. 2005.12.016(2006 ).
45 Sprogar, S., Volk, J., Drevensek, M. & Drevensek, G. The effects of TBC3214, a selective endothelin ETA receptor antagonist, on orthodontic tooth movement in rats. Eur J Orthod 29, 605-608, doi: 10.1093/ejo/cjm064 (2007).
46 Sprogar, S., Vaupotic, T., Cor, A., Drevensek, M. & Drevensek, G. The endothelin system mediates bone modeling in the late stage of orthodontic tooth movement in rats. Bone 43, 740-747, doi: 10.1016/j. bone. 2008.06.012(2008 ).
47 Meyer, K., Patra, T., Vijayamahantesh & Ray, R. SARS-COV-2 Spike Protein Induces Paracrine Senescence and Leukocyte Adhesion in Endothelial Cells. J Virol 95, e0079421, doi: 10.1128/JVI. 00794-21 (2021).
48 Evangelou, K. et al. Pulmonary infection by SARS-COV-2 induces senescence accompanied by an inflammatory phenotype in severe COVID-19: possible implications for viral mutagenesis. Eur Respir J 60, doi: 10.1183/13993003.02951-2021 (2022).
49 Lee, S. et al. Virus-induced senescence is driver and therapeutic target in COVID-19. Nature, doi: 10.1038/s41586-021-03995-1 (2021).
50 Camell, C. D. et al. Senolytics reduce coronavirus-related mortality in old mice. Science 373, doi: 10.1126/science. abe4832(2021 ).
51 Urata, R. et al. Senescent endothelial cells are predisposed to SARS-COV-2 infection and subsequent endothelial dysfunction. Sci Rep 12, 11855, doi: 10.1038/s41598-022-15976z (2022).
52 Olmos, G. et al. Hyperphosphatemia induces senescence in human endothelial cells by increasing endothelin-1 production. Aging Cell 16, 1300-1312, doi: 10.1111/acel. 12664(2017 ).
53 Abdul, Y., Karakaya, E., Chandran, R., Jamil, S. & Ergul, A. Endothelin A receptors contribute to senescence of brain microvascular endothelial cells. Can J Physiol Pharmacol 100, 1087-1096, doi: 10.1139/cjpp-2022-0071 (2022).
54 Bukowska, A. et al. Anti-inflammatory effects of endothelin receptor blockade in left atrial tissue of spontaneously hypertensive rats. Int J Cardiol Heart Vasc 42, 101088, doi: 10.1016/j. ijcha. 2022.101088(2022 ).
55 Guo, Q., Huang, J. A. & Fraidenburg, D. R. Bosentan as rescue treatment in refractory hypoxemia and pulmonary hypertension in a patient with ARDS and H7N9 influenza virus infection. Lung 192, 635-636, doi: 10.1007/s00408-014-9602-9 (2014).
56 Landazuri, N. et al. The Endothelin Receptor Antagonist Macitentan Inhibits Human Cytomegalovirus Infection. Cells 10, doi: 10.3390/cells10113072 (2021).
57 Scuri, P. et al. An unexpected recovery of patients with pulmonary arterial hypertension and SARS-COV-2 pneumonia: a case series. Pulm Circ 10, 2045894020956581,doi: 10.1177/2045894020956581 (2020).
58 Cambridge University Hospitals, N.H.S.F.T. mulTi-Arm Therapeutic Study in Pre-ICu Patients Admitted With Covid-19-Experimental Drugs and Mechanisms, <https: //ClinicalTrials. gov/show/NCT04393246> (2022).
59 Miedel, E. L. & Hankenson, F. C. Biology and Diseases of Hamsters. Laboratory Animal Medicine, 209-245, doi: 10.1016/B978-0-12-409527-4.00005-5 (2015).
60 Filep, J. G., Sirois, M.G., Rousseau, A., Fournier, A. & Sirois, P. Effects of endothelin-1 on vascular permeability in the conscious rat: interactions with platelet-activating factor. Br J Pharmacol 104, 797-804, doi: 10.1111/j. 1476-5381.1991. tb12509. x (1991).
61 Chu, H. et al. Comparative tropism, replication kinetics, and cell damage profiling of SARS-COV-2 and SARS-COV with implications for clinical manifestations, transmissibility, and laboratory studies of COVID-19: an observational study. Lancet Microbe 1, e14-e23, doi: 10.1016/S2666-5247 (20) 30004-5 (2020).
62 Chan, J. F. et al. A familial cluster of pneumonia associated with the 2019 novel coronavirus indicating person-to-person transmission: a study of a family cluster. Lancet 395, 514-523, doi: 10.1016/S0140-6736 (20) 30154-9 (2020).
63 Yuan, S. et al. Metallodrug ranitidine bismuth citrate suppresses SARS-COV-2 replication and relieves virus-associated pneumonia in Syrian hamsters. Nat Microbiol 5, 1439-1448, doi: 10.1038/s41564-020-00802-x (2020).
64 Yuan, S. et al. Pathogenicity, transmissibility, and fitness of SARS-COV- 2 Omicron in Syrian hamsters. Science 377, 428-433, doi: 10.1126/science. abn 8939 (2022).
65 Nair, A. B. &Jacob, S. A simple practice guide for dose conversion between animals and human. J Basic Clin Pharm 7, 27-31, doi: 10.4103/0976-0105.177703 (2016).
66 To, K. K. et al. Temporal profiles of viral load in posterior oropharyngeal saliva samples and serum antibody responses during infection by SARS-COV-2: an observational cohort study. Lancet Infect Dis 20, 565-574, doi: 10.1016/S1473-3099 (20) 30196-1 (2020).

1. A method of treating or preventing a skeletal sequelae of a coronavirus infection in a subject in need thereof, the method comprising administering a therapeutically effective amount of an endothelin receptor antagonist to the subject, with the proviso that the endothelin receptor antagonist is not co-administered with dapagliflozin or niclosamide.

2. The method of claim 1, wherein the subject does not suffer from pulmonary hypertension.

3. The method of claim 1, wherein the coronavirus infection is the result of a coronavirus selected from the group consisting of severe acute respiratory syndrome coronavirus (SARS-COV), severe acute respiratory syndrome coronavirus 2 (SARS-COV-2), Middle East respiratory syndrome-related coronavirus (MERS-COV), human coronavirus 229E (229E-CoV-2), human coronavirus NL63 (NL63-CoV), human coronavirus OC43 (OC43-CoV), human coronavirus HKU1 (HCoV-HKU1), and variants thereof.

4. The method of claim 1, wherein the coronavirus infection is the result of a severe acute respiratory syndrome coronavirus 2 (SARS-COV-2) and variants thereof.

5. The method of claim 1, wherein the endothelin receptor antagonist is a selective ETA receptor antagonist, a selective ETB receptor antagonist, or a dual ETA receptor and ETB receptor antagonist.

6. The method of claim 1, wherein the endothelin receptor antagonist is a dual ETA receptor and ETB receptor antagonist.

7. The method of claim 1, wherein the endothelin receptor antagonist is selected from the group consisting of sitaxsentan, ambrisentan, atrasentan, BQ-123, sparsentan, zibotentan, aprocitentan, edonentan, bosentan, macitentan, tezosentan, BQ-788, A192621, and mixtures thereof.

8. The method of claim 1, wherein the endothelin receptor antagonist is bosentan, macitentan, ambrisentan, or a mixture thereof.

9. The method of claim 1, wherein the endothelin receptor antagonist is macitentan.

10. The method of claim 1, wherein the coronavirus infection is the result of a severe acute respiratory syndrome coronavirus 2 (SARS-CoV-2) and the endothelin receptor antagonist is bosentan, macitentan, ambrisentan, or a mixture thereof.

11. The method of claim 1, wherein the endothelin receptor antagonist is administered to the subject in at least one of the coronavirus infection acute phase or coronavirus infection sub-acute phase.

12. The method of claim 1, wherein the skeletal sequelae is one or more of arthralgia, viral arthritis, osteopenia, osteochondral damage, and osteoporosis.

13. The method of claim 1, wherein the endothelin receptor antagonist is administered to the subject intraarticular injection or intraosseous injection.

14. The method of claim 1, wherein the coronavirus infection is the result of a severe acute respiratory syndrome coronavirus 2 (SARS-COV-2) and the endothelin receptor antagonist is macitentan.

15. The method of claim 14, wherein macitentan is administered to the subject in at least one of SARS-COV-2 acute phase or coronavirus infection sub-acute phase.

16. The method of claim 15, wherein the skeletal sequelae is one or more of arthralgia, viral arthritis, osteopenia, osteochondral damage, and osteoporosis.
